(19) United States
(12) Patent Application Publication
(10) Pub. No.: US 2024/0374612 A1
(43) Pub. Date: Nov. 14, 2024

(54) IMIDAZOBENZODIAZEPINES FOR TREATMENT OF COGNITIVE AND MOOD SYMPTOMS

(71) Applicants: UWM Research Foundation, Inc., Milwaukee, WI (US); Centre for Addiction and Mental Health, Toronto (CA)

(72) Inventors: Thomas Damien Prevot, Toronto (CA); Michael Jeffrey Marcotte, Toronto (CA); Etienne Laurent Sibille, Mono (CA); James M. Cook, Milwaukee, WI (US); Guangan Li, Shenzhen, Guangdong Province (CN); Prithu Mondal, Milwaukee, WI (US); Md Yeunus Mian, Milwaukee, WI (US); Farjana Rashid, Middleton, WI (US)

(21) Appl. No.: 18/690,342

(22) PCT Filed: Sep. 8, 2022
(86) PCT No.: PCT/US2022/042832

§ 371 Date: Mar. 8, 2024

Related U.S. Application Data
(60) Provisional application No. 63/334,978, filed on Apr. 26, 2022.
(60) Provisional application No. 63/241,877, filed on Sep. 8, 2021.

(51) Int. Cl. A61K 31/5517; A61P 25/00; C07D 487/04

(52) U.S. Cl. CPC A61K 31/5517; A61P 25/00; C07D 487/04

(57) ABSTRACT

4H-Benzo[f]imidazo [1,5-a][1,4]diazepines substituted in the 3-position with an amide or oxadiazole and their pharmaceutical compositions display efficacy to enhance cognition and may have utility in the treatment of cognitive deficiencies, such as in neurodegenerative and neuropsychiatric disorders.

### CROSS-REFERENCE TO RELATED APPLICATIONS

This application claims the benefit of U.S. Provisional Application No. 63/241,877 filed on Sep. 8, 2021, and U.S. Provisional Application No. 63/334,978 filed on Apr. 26, 2022, the entire contents of which are incorporated by reference herein.

### TECHNICAL FIELD

Imidazobenzodiazepine amides and oxadiazoles and their pharmaceutical compositions are useful to enhance cognition for treatment of cognitive deficiencies in neurodegenerative and neuropsychiatric disorders.

### BACKGROUND

According to the World Health Organization, major depressive disorder (MDD) is a leading cause of disability, affecting 350 million people worldwide. It is a complex disorder with symptoms that include low affect, anhedonia, anxiety, rumination, appetite changes, sleep disturbances, and cognitive impairments. Its early onset and chronic nature have serious consequences for lost education and unemployment. It has an estimated annual and lifelong prevalence of 5.3% and 13.2% respectively, with a high rate of recurrence, higher prevalence in women, and a 37% rate of heritability. MDD is the leading cause of years lost due to disability both in developing and developed countries, and in both women and men. The burden of depression is growing. Treatment-resistant depression, a severe and chronic form of the illness, has an estimated prevalence of 1-3% of the population at any given time, a proportion that is greater than schizophrenia and bipolar disorder cases combined. Despite an unacceptable burden on affected individuals, their family and society, pharmaceutical companies have mostly withdrawn from developing new antidepressant drugs.

Cognitive impairments are part of the comorbid symptoms that develop alongside anxiety, anhedonia, sleep disturbance and other deficits. Cognitive dysfunction refers to deficits in attention, visual and auditory processing, short term and working memory, motor function, learning and memory processes. Despite overwhelming consensus on the importance of cognitive impairment in depression, there is no conclusion regarding the full profile of cognitive impairment in depression. Cognitive impairments may be a primary dysfunction in MDD and several other core symptoms may act as mediators of cognitive dysfunction. Current antidepressant medications are all derived from approaches and modes of action that were discovered by chance over 50 years ago. These drugs act predominantly on the monoamine (serotonin and norepinephrine) systems. They often take weeks to achieve therapeutic effects, and subjects experience poor response, low remission rate (˜50%) and considerable side-effects. Moreover, available antidepressant are not designed to treat cognitive impairment, and in some case (like some benzodiazepine), their positive effects on some dimensions of the illness (anxiety, anhedonia) are counterbalanced by negative side effects affecting cognition. Furthermore, clinical studies have demonstrated that cognitive deficits are still detected even in periods of remission from mood symptoms. Hence, developing antidepressants that can potentially rescue the cognitive dysfunction as well as the emotional and motivational symptoms seem critical for future treatment of MDD.

The barriers to new antidepressant drug development are multiple, starting with a paucity of knowledge on disease mechanisms and of targets that are informed by the primary pathology of the illness. Accordingly, there is currently little effort made toward rational drug design or for developing biological diagnostic and therapeutic markers for personalized treatments.

### SUMMARY

In one aspect, the invention provides a compound selected from the group consisting of:

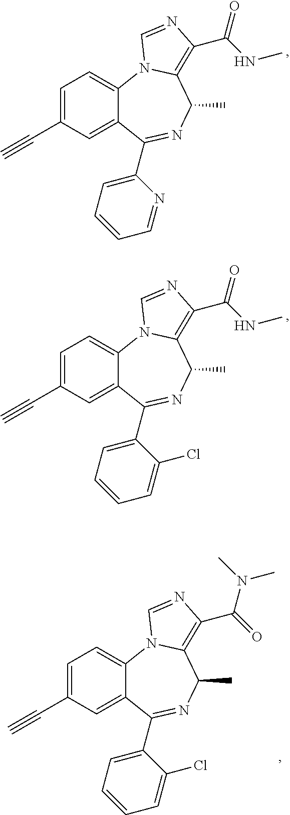

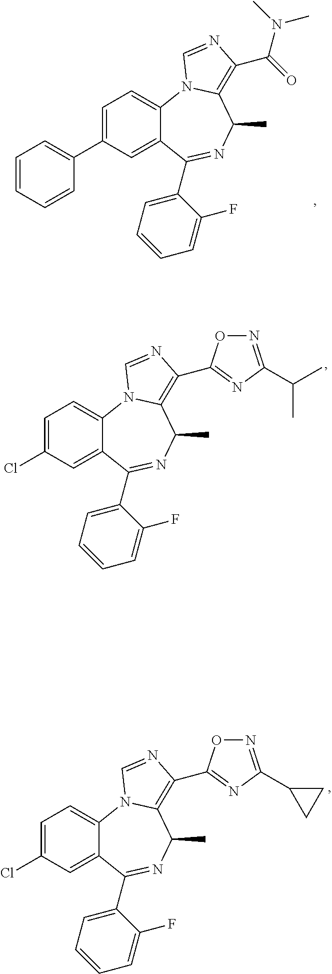 [SMILES: C[C@H]1N=C(c2ccccc2F)c2cc(-c3ccccc3)ccc2-n2cnc(C(=O)N(C)C)c21 ; CC(C)c1noc(-c2ncn3c2[C@@H](C)N=C(c2ccccc2F)c2cc(Cl)ccc2-3)n1 ; C[C@H]1N=C(c2ccccc2F)c2cc(Cl)ccc2-n2cnc(-c3nc(C4CC4)no3)c21]

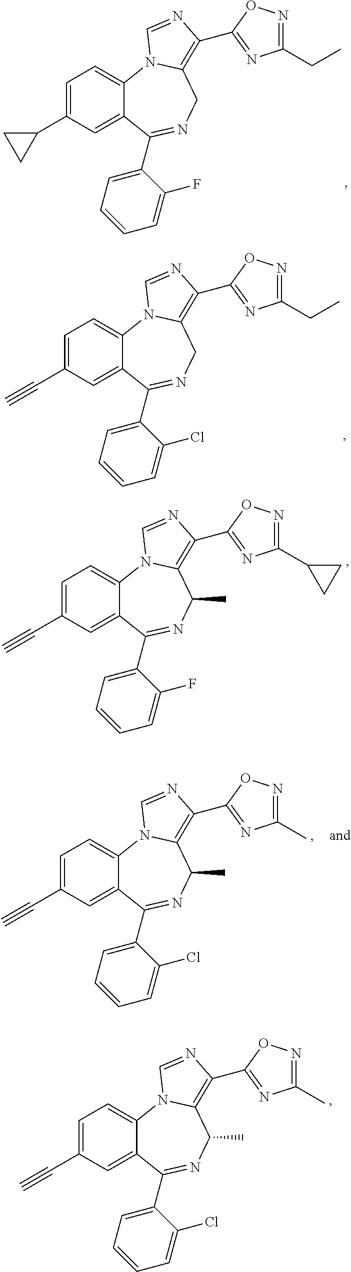

or a pharmaceutically acceptable salt thereof.

Another aspect of the invention provides pharmaceutical compositions comprising a compound selected from the group consisting of

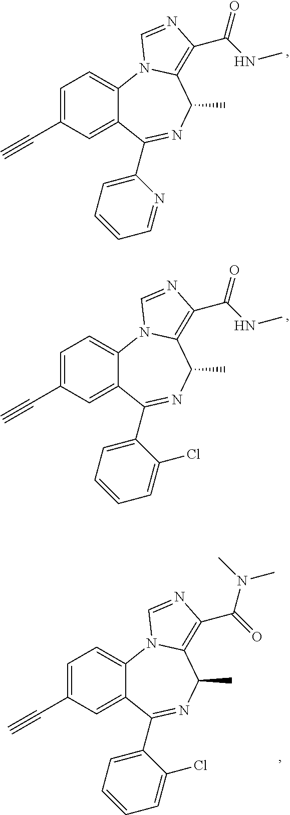

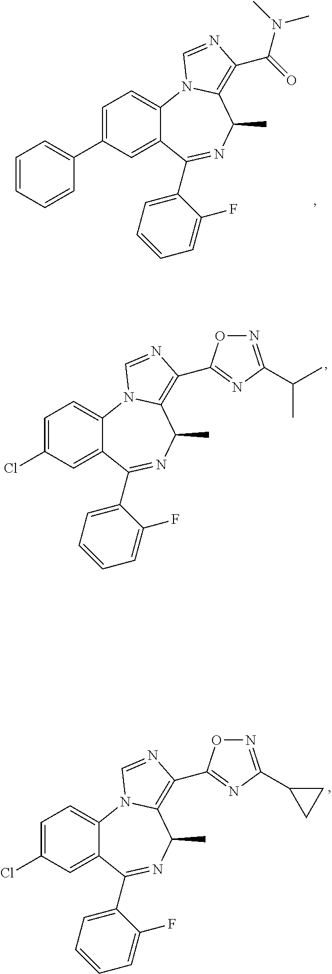 [SMILES: C[C@H]1N=C(c2ccccc2F)c2cc(-c3ccccc3)ccc2-n2cnc(C(=O)N(C)C)c21 ; CC(C)c1noc(-c2ncn3c2[C@@H](C)N=C(c2ccccc2F)c2cc(Cl)ccc2-3)n1 ; C[C@H]1N=C(c2ccccc2F)c2cc(Cl)ccc2-n2cnc(-c3nc(C4CC4)no3)c21]

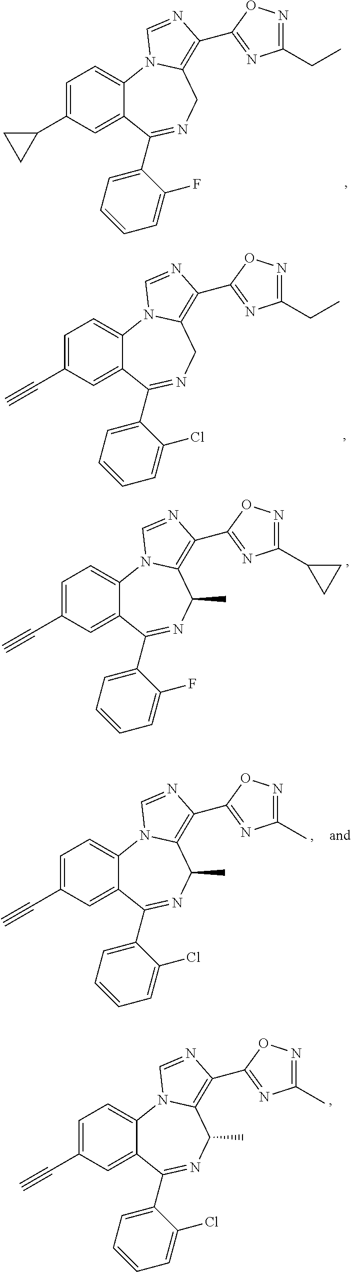

and a pharmaceutically acceptable carrier.

In another aspect, the invention provides a compound, or a pharmaceutically acceptable salt thereof, for use in a method of enhancing cognition, wherein the compound is selected from the group consisting of

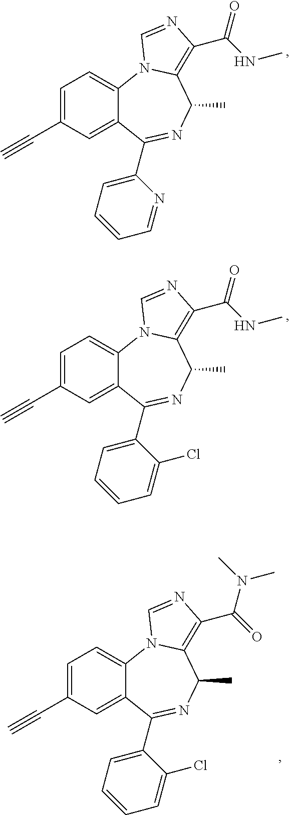

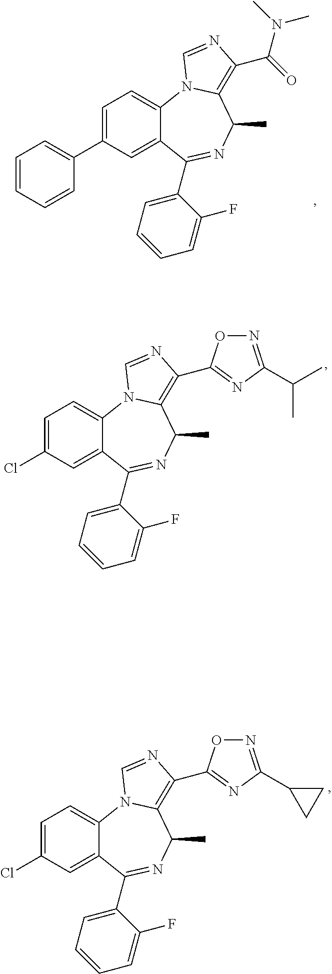 [SMILES: C[C@H]1N=C(c2ccccc2F)c2cc(-c3ccccc3)ccc2-n2cnc(C(=O)N(C)C)c21 ; CC(C)c1noc(-c2ncn3c2[C@@H](C)N=C(c2ccccc2F)c2cc(Cl)ccc2-3)n1 ; C[C@H]1N=C(c2ccccc2F)c2cc(Cl)ccc2-n2cnc(-c3nc(C4CC4)no3)c21]

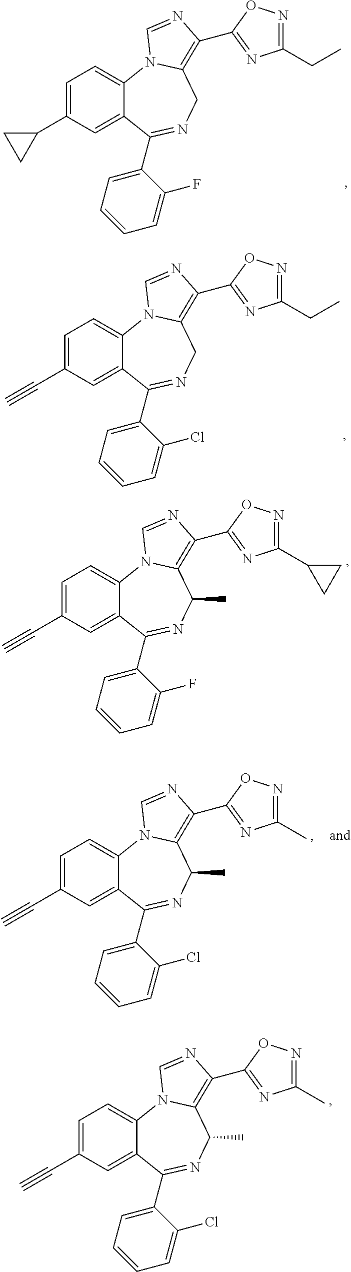

In another aspect, the invention provides the use of a compound, or a pharmaceutically acceptable salt thereof, in the manufacture of a medicament for enhancing cognition, wherein the compound is selected from the group consisting of

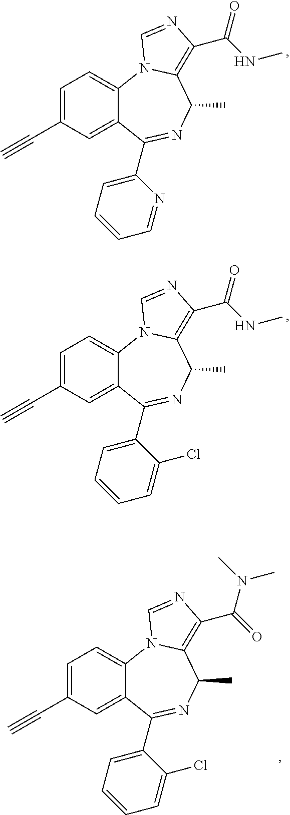

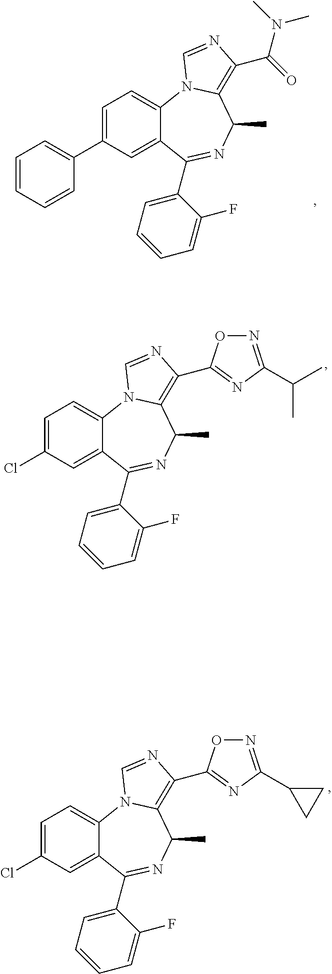 [SMILES: C[C@H]1N=C(c2ccccc2F)c2cc(-c3ccccc3)ccc2-n2cnc(C(=O)N(C)C)c21 ; CC(C)c1noc(-c2ncn3c2[C@@H](C)N=C(c2ccccc2F)c2cc(Cl)ccc2-3)n1 ; C[C@H]1N=C(c2ccccc2F)c2cc(Cl)ccc2-n2cnc(-c3nc(C4CC4)no3)c21]

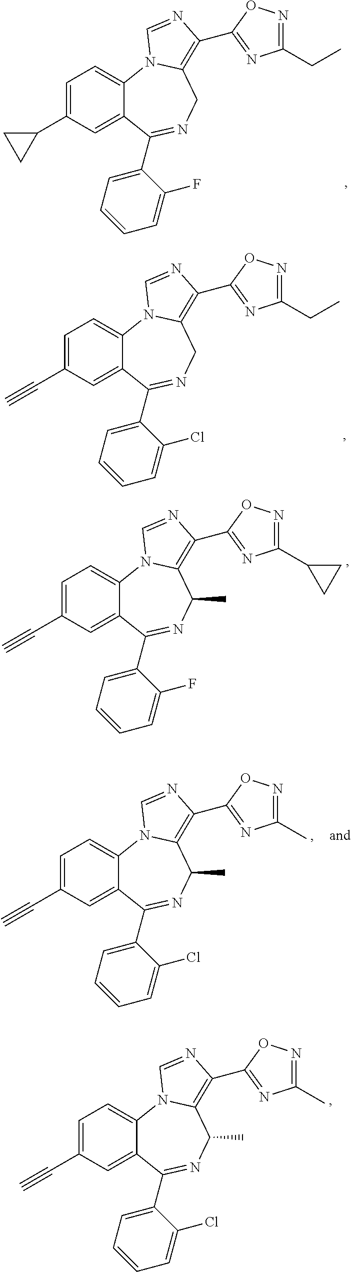

Other aspects of the disclosure will become apparent by consideration of the detailed description and accompanying drawings.

### BRIEF DESCRIPTION OF THE DRAWINGS

FIG. 1 shows that chronic restraint stress (CRS) induces significant reduction of alternation rate (p<0.001) compared to Control Vehicle mice. This effect is reversed by acute administration of GL-III-68, 10 mg/kg, 30 minutes prior to testing. This result suggests a pro-cognitive effect of GL-III-68, reversing working memory deficits.

FIG. 2A shows a significant difference between groups (ANOVA: F(2,12)=7.3, p<0.01). Fisher's PLSD post hoc analysis shows that chronic restraint stress (CRS) induces significant reduction of alternation rate (p<0.01) compared to Control Vehicle mice. This effect is reversed (p<0.05) by acute administration of MYM-III-29, 10 mg/kg, 30 minutes prior to testing. This result suggests a pro-cognitive effect of MYM-III-29, reversing working memory deficits.

FIG. 2B shows the results of mice tested in the Y Maze, after CRS and acute treatment with MYM-III-29 at 3 mg/kg. Results show no significant difference between groups (ANOVA: F(2,11)=2.7, p=0.1).

FIG. 3 shows a significant effect of 10 mg/kg (i.p.) MYM-III-29 in the elevated plus maze, increasing distance travelled in the open arms (t-test: p<0.05). This result suggests a potential anxiolytic effect of MYM-III-29.

FIG. 4 shows that chronic restraint stress (CRS) induces significant reduction of alternation rate (p<0.0001) compared to Control Vehicle mice. This effect is reversed by acute administration of FR-III-17, 0.3 mg/kg ($$ p<0.01), 1 mg/kg ($ p<0.05) or 3 mg/kg ($ p<0.05), 30 minutes prior to testing. This result suggests a pro-cognitive effect of FR-III-17, reversing working memory deficits.

FIG. 5A shows a significant difference between groups (ANOVA: F(2,11)=2, p<0.05). Fisher's PLSD post hoc analysis shows that chronic restraint stress (CRS) induces significant reduction of alternation rate (p<0.05) compared to Control Vehicle mice. This effect is reversed (trend: p=0.1) by acute administration of PM-II-26, 30 mg/kg, 30 minutes prior to testing. This result suggests a pro-cognitive effect of PM-II-26, reversing working memory deficits.

FIG. 5B shows a significant difference between groups (ANOVA: F(2,7)=19.6, p<0.001). Fisher's PLSD post hoc analysis shows that chronic restraint stress (CRS) induces significant reduction of alternation rate (p<0.001) compared to Control Vehicle mice. This effect is reversed (p<0.001) by acute administration of PM-II-26, 10 mg/kg, 30 minutes prior to testing. This result suggests a pro-cognitive effect of PM-II-26, reversing working memory deficits.

FIG. 6 shows that chronic restraint stress (CRS) induces significant reduction of alternation rate (p<0.0001) compared to Control Vehicle mice. This effect is reversed by acute administration of MYM-III-41, 5 mg/kg (p<0.01) and 10 mg/kg (p<0.05), 30 minutes prior to testing. This result suggests a pro-cognitive effect of MYM-III-41, reversing working memory deficits.

FIG. 7 shows a significant difference between groups (ANOVA: F(3,14)=9.7, p<0.001). Fisher's PLSD post hoc analysis shows that chronic restraint stress (CRS) induces significant reduction of alternation rate (p<0.01) compared to Control Vehicle mice. This effect is reversed by acute administration of GL-IV-03, 3 mg/kg (p<0.01) and 30 mg/kg (p<0.01), 30 minutes prior to testing. This result suggests a pro-cognitive effect of GL-IV-03, reversing working memory deficits.

FIG. 8 shows a significant difference between groups (ANOVA: F(2,28)=5.3, p<0.05). Fisher's PLSD post hoc analysis shows that chronic restraint stress (CRS) induces significant reduction of alternation rate (p<0.01) compared to Control Vehicle mice. This effect is reversed (p<0.05) by acute administration of GL-1-65, 10 mg/kg, 30 minutes prior to testing. This result suggests a pro-cognitive effect of GL-1-65, reversing working memory deficits.

FIG. 9 shows a significant improvement of alternation rate in old mice receiving acute administration of GL-1-65, 10 mg/kg, 30 minutes prior to testing (t-test: p<0.05 compared to old mice receiving vehicle).

FIG. 10 shows that chronic restraint stress (CRS) induces significant reduction of alternation rate (p<0.001) compared to Control Vehicle mice. This effect is reversed by acute administration of GL-III-60, 10 mg/kg ($$ p<0.01) and 30 mg/kg ($ p<0.05), 30 minutes prior to testing. This result suggests a pro-cognitive effect of GL-III-60, reversing working memory deficits.

FIG. 11 shows a significant difference between groups (ANOVA: F(2,11)=4.4, p<0.05). Fisher's PLSD post hoc analysis shows that chronic restraint stress (CRS) induces significant reduction of alternation rate (p<0.05) compared to Control Vehicle mice. This effect is reversed (trend p=0.06) by acute administration of GL-II-33, 10 mg/kg, 30 minutes prior to testing. This result suggests a pro-cognitive effect of GL-II-33, reversing working memory deficits.

FIG. 12 shows that chronic restraint stress (CRS) induces significant reduction of alternation rate (p<0.001) compared to Control Vehicle mice. This effect is reversed by acute administration of MYM-III-43, 3 mg/kg ($ p<0.05), 10 mg/kg ($$ p<0.01), and 30 mg/kg ($$ p<0.01), 30 minutes prior to testing. This result suggests a pro-cognitive effect of MYM-III-43, reversing working memory deficits.

FIG. 13 shows a significant difference between groups (ANOVA: F(2,11)=10, p<0.01). Fisher's PLSD post hoc analysis shows that chronic restraint stress (CRS) induces significant reduction of alternation rate (p<0.05) compared to Control Vehicle mice. This effect is reversed by acute administration of PM-II-84E, 30 mg/kg (p<0.05), 30 minutes prior to testing. This result suggests a pro-cognitive effect of PM-II-84E, reversing working memory deficits.

FIG. 14 shows that chronic restraint stress (CRS) induces significant reduction of alternation rate (p<0.001) compared to Control Vehicle mice. This effect is reversed by acute administration of MYM-IV-47, 10 mg/kg ($$<0.01) and 30 mg/kg ($ p<0.05), 30 minutes prior to testing. This result suggests a pro-cognitive effect of MYM-IV-47, reversing working memory deficits.

FIG. 15 shows a significant effect of 10 mg/kg (i.p.) PM-II-84E in the elevated plus maze, increasing time spent in the open arms (t-test: p<0.01). This result suggests a potential anxiolytic effect of PM-II-84E.

FIG. 16 shows 10 mg/kg and 30 mg/kg (i.p.) MYM-V-28 in the Y-maze spatial alternation task for working memory significantly improved alternation rate ($ p<0.05) in comparison to control (CRS Vehicle).

FIG. 17 shows 10 mg/kg (i.p.) PM-III-57R in the Y-maze spatial alternation task for working memory significantly improved alternation rate ($$ p<0.01) in comparison to control (CRS Vehicle).

FIG. 18 shows 10 mg/kg (i.p.) FR-PM-III-57S in the Y-maze spatial alternation task for working memory significantly improved alternation rate ($ p<0.05) in comparison to control (CRS Vehicle).

FIG. 19 shows MYM-IV-46 at 10 mg/kg improved alternation rate ($$<0.01) in the Y-Maze alternation task for working memory in comparison to control (CRS Vehicle).

FIG. 20 shows FR-III-17 (3 mg/kg, i.p., 30 min before testing) and vehicle controls were tested in the elevated plus maze for anxiety like behaviors. t-tests found FR-III-17 significantly increase % time, % distance traveled and % entries in the open arms (* p<0.05, ** P<0.01) (n=6 per group), suggesting anxiolytic properties.

FIG. 21 shows electrophysiological characterization of GL-III-68 in cells transfected with human α1β2γ3, α2β2γ3, α3β2γ3, α4β2γ3, or α5β2γ3 GABA_{A} receptors.

### DETAILED DESCRIPTION

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various ways.

Improvement in medical interventions and living conditions have dramatically increased the average human lifespan. As a result, a growing number of people are suffering from neurodegenerative diseases, including dementia. 46.8 million people worldwide lived with dementia in 2015 and this number is expected to double every 20 years, reaching 74.7 million in 2030 and 131.5 million in 2050. A majority of people with dementia suffer from Alzheimer's disease (AD). AD is a fatal disease that affects cognitive, emotional and behavioral aspects of a person's life. Cognitive symptoms include negative effects on a person's ability to make decisions, perform simple tasks, or follow a conversation. Anticholinergic drugs are used for the cognitive deficits in AD but with very limited success. Mood symptoms in AD include apathy, loss of interest, low affect, anxiety and withdrawal. AD is exerting an increasing personal, societal and economic burden of AD, namely $604 billion in 2013, predicted to surpass $2 trillion in 2050, not counting the burden of prodromal (mild cognitive impairment) and high risk factor (aging and depression) states. This burden is compounded by the recent failures of clinical trials aimed at the classical neuropathologies of AD (i.e. amyloid plaques and tangles).

Recent progress in uncovering the biological bases of brain disorders demonstrates a significant sharing and continuum of cellular and molecular pathologies across the classical categorical diagnostics of diseases. Accordingly the GABA-related pathology that is described in this document and that is targeted by compounds with agonist or positive allosteric activities at alpha5-containing GABA-_{A} receptors has been reported across brain disorders from neuropsychiatric (e.g., depression, schizophrenia) to neurodegenerative (e.g. AD) disorders. Hence the novel therapeutic strategies could be directed at specific disorders as well as symptom dimensions (e.g. cognition, mood) across disorders, from neuropsychiatric (e.g., depression, schizophrenia) to neurodegenerative (e.g. AD) disorders.

The molecular and cellular pathology of brain disorders has been investigated using unbiased genomic approaches in human postmortem brains; genetic and environmental mouse models have been used to investigate causal links between the pathologies identified in the human brain and mood and cognitive regulatory mechanisms in rodents; targets have been identified within those causal pathological modules and have performed preliminary preclinical studies to support the value of these targets as novel therapeutic strategies. This has led to the GABA system and to the alpha5 subunit containing GABA_{A} receptor as a novel target for cognitive remediation antidepressant/anxiolytic treatment.

More specifically, converging evidence has long suggested a role for the inhibitory GABA system in depression. Recent evidence suggests a specific cellular origin for those changes (Guilloux et al, Molecular Psychiatry, 2012, 17, 1130-1142; Tripp et al, Am J Psychiatry, 2012, 169, 1194-1202). These findings have been integrated into a model, linking GABA-related biochemical, cellular and brain region findings with psychological concepts and symptom dimensions in depression (Northoff & Sibille, Molecular Psychiatry, 2014, 19, 966-977). In this model, deficits in somatostatin-positive (SST+) GABA neurons that regulate excitatory input onto the dendrites of pyramidal cells translate into altered information processing by local cell circuits, and result in altered activity of key brain regions (frontal and cingulate cortices) and neural networks (default-mode and executive networks). In turn these integrated biological deficits surface as anhedonia (lack of experiencing pleasure) and increased negative self-focus (rumination, suicidality), two central features of depression.

Based on the inspection of the molecular components of the cell-specific link between SST+ GABA and pyramidal neurons, it is proposed that the alpha 5 subunit of the GABA_{A} receptor is a logical target to remediate the molecular pathology of depression and to potentially exert pro-cognitive and antidepressant-like activity. The different alpha subunits of GABA_{A} receptors determine the localization of these receptors across cellular compartments. Alpha 5-containing GABA_{A} receptors are located on dendrites of pyramidal cells, opposite from SST+ GABA neuronal terminals; hence they mediate the function of SST+ GABA neurons. The GABA_{A} receptor alpha subunits are the main targets of benzodiazepine-like compounds. These compounds have sedative, anxiolytic and anticonvulsant effects. This broad activity is due to their non-specific targeting of several alpha subunits. This pan-alpha subunit activity has considerably limited their therapeutic potential, due to sedation, tolerance and cognitive side-effects. Recent anatomical, genetic and functional characterization of the various GABA_{A}R alpha subunits has raised hopes that the selective targeting of specific subunits will uncover novel therapeutic opportunities for neuropsychiatric disorders.

Here a novel pro-cognitive and antidepressant modality is proposed which is informed by the primary molecular pathology of depression. The primary target is the inhibitory GABA_{A} receptor Alpha5 subunit, the pharmacological effect is positive allosteric modulation (Alpha5-PAM), and the therapeutic indication is for depression and other disorders that share mood and cognitive deficits, potentially focusing on the cognitive and rumination core symptoms. The rationale for choosing this target is based on (1) the findings using human postmortem brain samples suggesting reduced function at the GABA_{A}-Alpha5-containing synapse in depression, (2) a large body of research suggesting altered GABA function in depression, and (3) the preclinical rodent studies showing antidepressant effects of Alpha5-PAM (Piantodosi et al, Frontiers in Pharmacology, 2016, 7, 446) and pro-cognitive effects. Note that recent findings also suggest that reducing the function of alpha5-containing GABA-_{A} receptors may exert antidepressant activity (Fischell et al, Neuropsychopharmacology, 2015, 40, 2499-2509). This suggests a putative inverted U-shape effect for alpha5-GABA-A receptor function, where both high and low function may have therapeutic potential. However reducing the function of alpha5-containing GABA-_{A} receptors is notably predicted to worsen the primary pathology observed in brain disorders (i.e. reduced SST cell function), hence it is potentially associated with higher risk for long-term detrimental effects.

There is strong evidence of reduced expression and function of somatostatin (SST)-positive inhibitory GABA neurons in neurological disorders, including AD, schizophrenia, bipolar depression, and major depression (Lin and Sibille, Frontiers Pharmacology, 2013, 4, 110). SST-positive GABA neurons are a subtype of inhibitory neurons which are characterized by inhibiting the dendritic compartment of glutamatergic pyramidal neurons, the main excitatory cells in the brain, Signaling through SST neurons regulate information and neural processes and has been specifically implicated in regulating cognition and mood. The main function of SST-positive neurons is mediated by the neurotransmitter GABA and by a specific subtype of GABA_{A} receptors which contain the alpha5 subunit. Alpha5-containing GABA_{A} receptors are localized on the dendrites of pyramidal cells, the cellular compartment targeted by SST-positive neurons. Hence, the deficits in SST positive cells that is observed across neurological disorders is postulated to result in reduced signaling though Alpha5-containing GABA_{A} receptors. Increasing Alpha5-containing GABA_{A} receptor signaling may therefore have therapeutic value for cognitive and mood symptoms across brain disorders, and specifically in AD and MDD.

### Definitions

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein, Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987; the entire contents of each of which are incorporated herein by reference.

In accordance with a convention used in the art, the group:

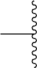

is used in structural formulas herein to depict the bond that is the point of attachment of the moiety or substituent to the core or backbone structure.

In the context of treating a disorder, the term “effective amount” as used herein refers to an amount of the compound or a composition comprising the compound which is effective, upon single or multiple dose administrations to a subject, in treating a cell, or curing, alleviating, relieving or improving a symptom of the disorder in a subject. An effective amount of the compound or composition may vary according to the application. In the context of treating a disorder, an effective amount may depend on factors such as the disease state, age, sex, and weight of the individual, and the ability of the compound to elicit a desired response in the individual. In an example, an effective amount of a compound is an amount that produces a statistically significant change in a given parameter as compared to a control, such as in cells (e.g., a culture of cells) or a subject not treated with the compound.

The term “subject” as used herein refers to mammals, such as humans, cats, dogs, horses, cattle, etc.

It is specifically understood that any numerical value recited herein (e.g., ranges) includes all values from the lower value to the upper value, i.e., all possible combinations of numerical values between the lowest value and the highest value enumerated are to be considered to be expressly stated in this application. For example, if a concentration range is stated as 1% to 50%, it is intended that values such as 2% to 40%, 10% to 30%, or 1% to 3%, etc., are expressly enumerated in this specification. These are only examples of what is specifically intended.

### Compounds

Compounds useful in the invention are set forth in the following numbered embodiments. The first embodiment is denoted E1, another embodiment is denoted E2 and so forth.

E1 A compound selected from the group consisting of:

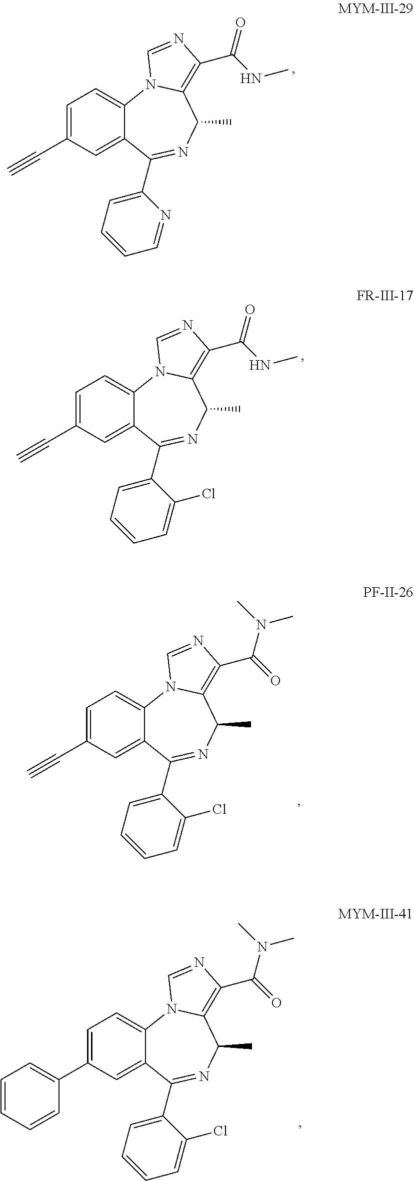

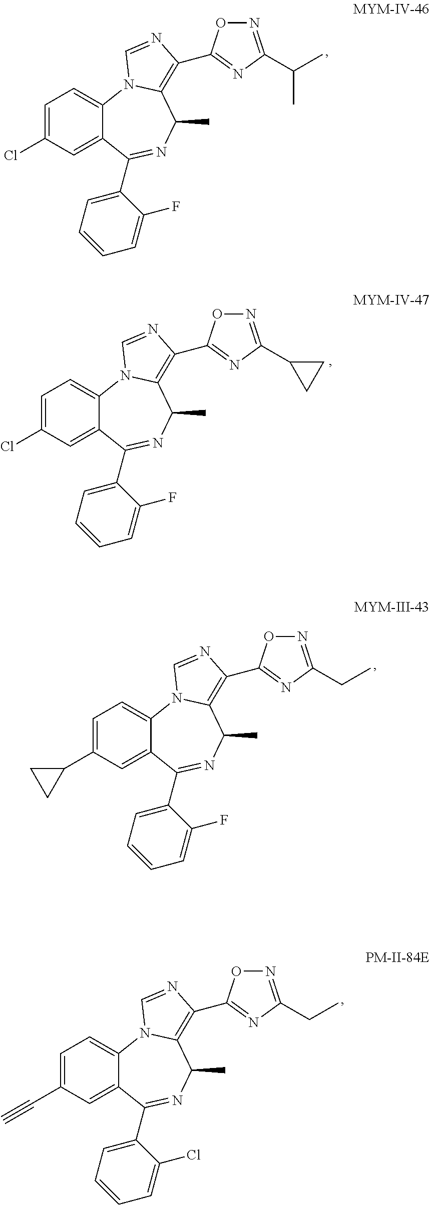

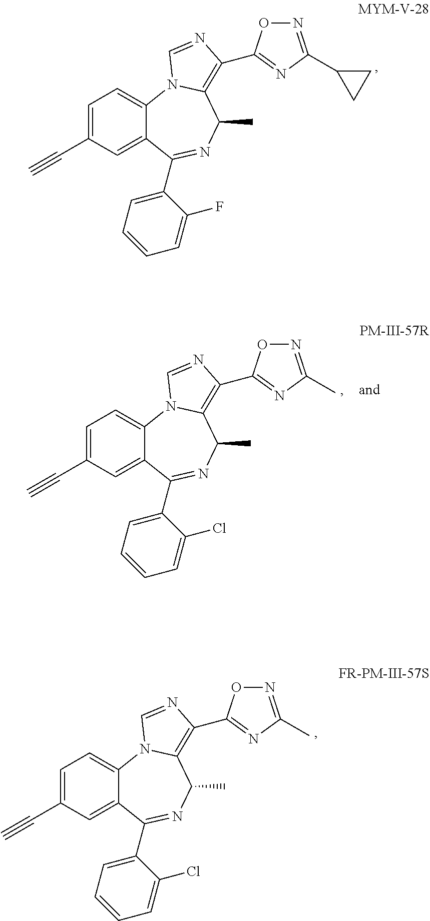

or a pharmaceutically acceptable salt thereof.

E1.1. A compound selected from the group consisting of:

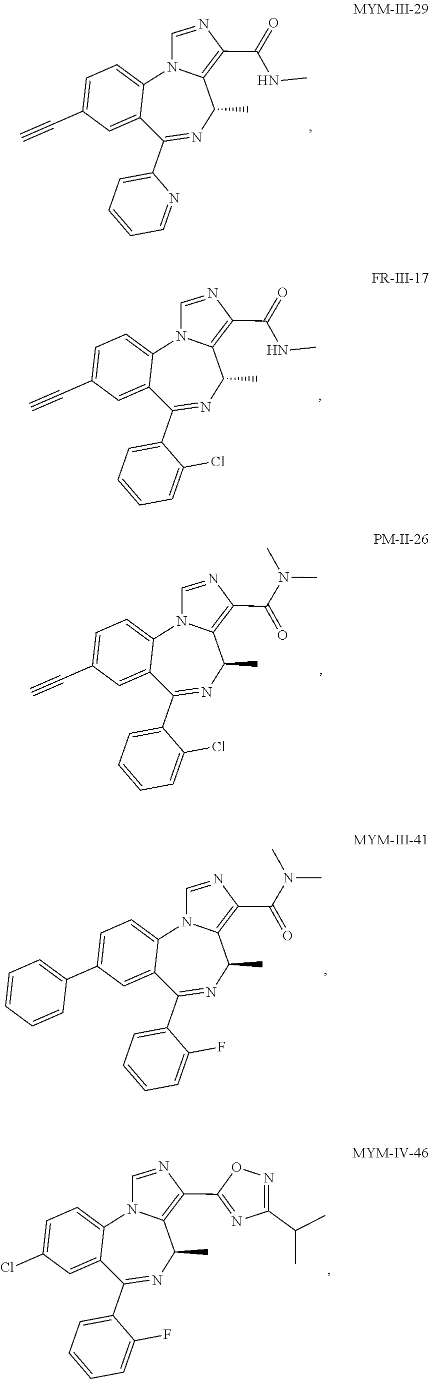

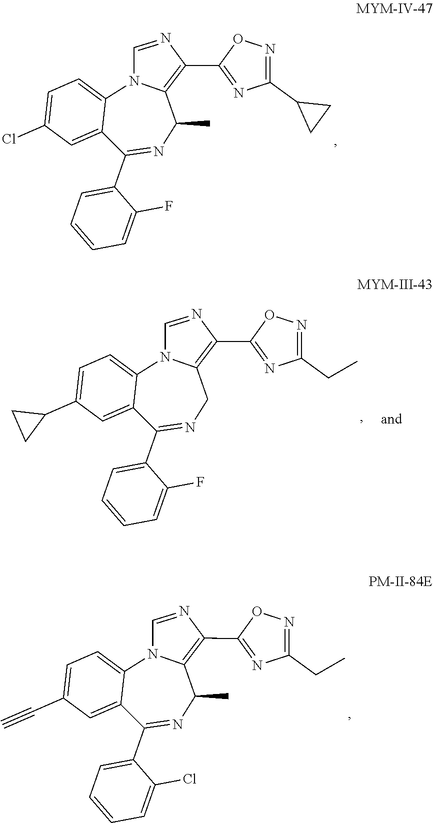

or a pharmaceutically acceptable salt thereof.

E2. The compound of E1, or a pharmaceutically acceptable salt thereof, wherein the compound is

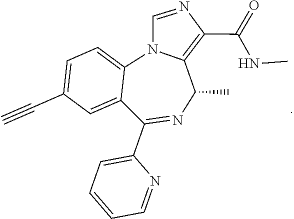 [SMILES: C#Cc1ccc2c(c1)C(c1ccccn1)=N[C@@H](C)c1c(C(=O)NC)ncn1-2]

E3. The compound of E1, or a pharmaceutically acceptable salt thereof, wherein the compound is

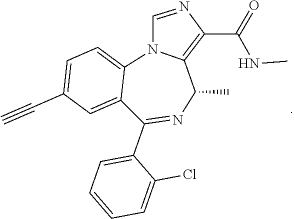 [SMILES: C#Cc1ccc2c(c1)C(c1ccccc1Cl)=N[C@@H](C)c1c(C(=O)NC)ncn1-2]

E4. The compound of E1, or a pharmaceutically acceptable salt thereof, wherein the compound is

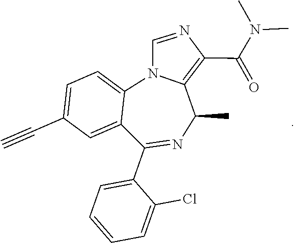 [SMILES: C#Cc1ccc2c(c1)C(c1ccccc1Cl)=N[C@H](C)c1c(C(=O)N(C)C)ncn1-2]

E5. The compound of E1, or a pharmaceutically acceptable salt thereof, wherein the compound is

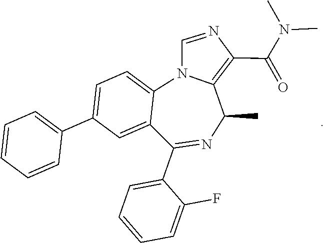 [SMILES: C[C@H]1N=C(c2ccccc2F)c2cc(-c3ccccc3)ccc2-n2cnc(C(=O)N(C)C)c21]

E6. The compound of E1, or a pharmaceutically acceptable salt thereof, wherein the compound is

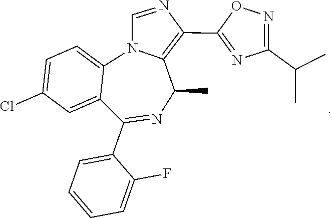 [SMILES: CC(C)c1noc(-c2ncn3c2[C@@H](C)N=C(c2ccccc2F)c2cc(Cl)ccc2-3)n1]

E7. The compound of E1, or a pharmaceutically acceptable salt thereof, wherein the compound is

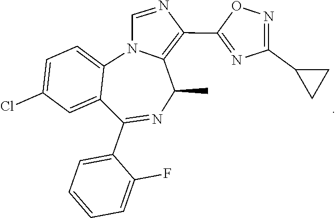 [SMILES: C[C@H]1N=C(c2ccccc2F)c2cc(Cl)ccc2-n2cnc(-c3nc(C4CC4)no3)c21]

E8. The compound of E1, or a pharmaceutically acceptable salt thereof, wherein the compound is

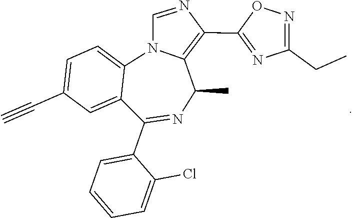 [SMILES: C#Cc1ccc2c(c1)C(c1ccccc1Cl)=N[C@H](C)c1c(-c3nc(CC)no3)ncn1-2]

E9. The compound of E1, or a pharmaceutically acceptable salt thereof, wherein the compound is

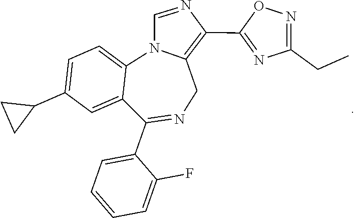 [SMILES: CCc1noc(-c2ncn3c2CN=C(c2ccccc2F)c2cc(C4CC4)ccc2-3)n1]

E10. The compound of E1 or a pharmaceutically acceptable salt thereof, wherein the compound is

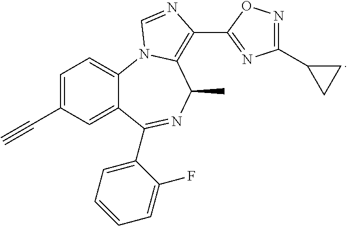 [SMILES: C#Cc1ccc2c(c1)C(c1ccccc1F)=N[C@H](C)c1c(-c3nc(C4CC4)no3)ncn1-2]

E11. The compound of E1, or a pharmaceutically acceptable salt thereof, wherein the compound is

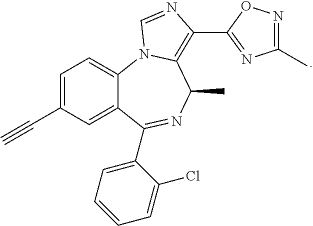 [SMILES: C#Cc1ccc2c(c1)C(c1ccccc1Cl)=N[C@H](C)c1c(-c3nc(C)no3)ncn1-2]

E12. The compound of E1, or a pharmaceutically acceptable salt thereof, wherein the compound is

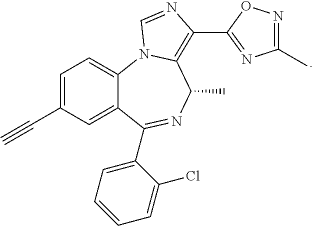 [SMILES: C#Cc1ccc2c(c1)C(c1ccccc1Cl)=N[C@@H](C)c1c(-c3nc(C)no3)ncn1-2]

E13. The compound of any of E1-E8 or E10-E12, or a pharmaceutically acceptable salt thereof, wherein the compound is substantially enantiomerically pure.

E14. A pharmaceutical composition comprising the compound of any of E1-E13, or a pharmaceutically acceptable salt thereof, and a pharmaceutically acceptable carrier.

E15. A method of enhancing cognition comprising administering to a subject in need thereof a therapeutically effective amount of the compound of any of E1-E13, or the pharmaceutical composition of E14, or a compound selected from the group consisting of:

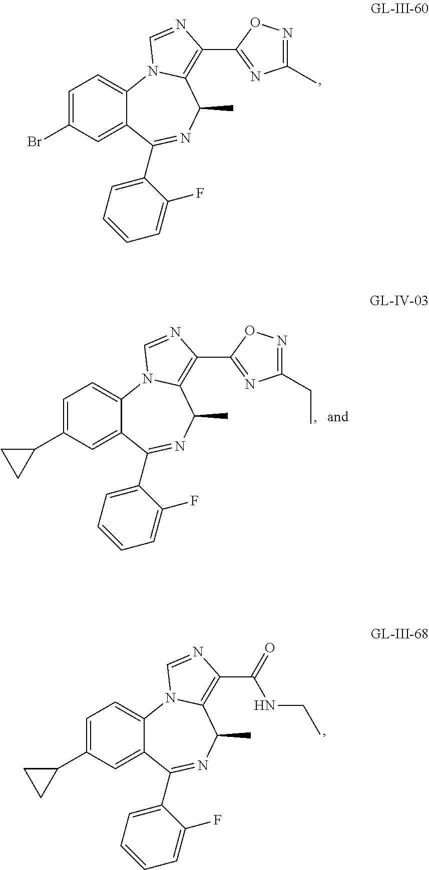 [SMILES: Cc1noc(-c2ncn3c2[C@@H](C)N=C(c2ccccc2F)c2cc(Br)ccc2-3)n1 ; CCc1noc(-c2ncn3c2[C@@H](C)N=C(c2ccccc2F)c2cc(C4CC4)ccc2-3)n1 ; CCNC(=O)c1ncn2c1[C@@H](C)N=C(c1ccccc1F)c1cc(C3CC3)ccc1-2]

or a pharmaceutically acceptable salt or composition thereof.

In the compounds of the invention, any “hydrogen” or “H,” whether explicitly recited or implicit in the structure, encompasses hydrogen isotopes ¹H (protium) and ²H (deuterium).

A compound can be in the form of a salt, e.g., a pharmaceutically acceptable salt. The term “pharmaceutically acceptable salt” includes salts of the active compounds that are prepared with relatively nontoxic acids or bases, depending on the particular substituents found on the compounds. Suitable pharmaceutically acceptable salts of the compounds of this disclosure include acid addition salts which may, for example, be formed by mixing a solution of the compound according to the disclosure with a solution of a pharmaceutically acceptable acid such as hydrochloric acid, sulfuric acid, methanesulfonic acid, fumaric acid, maleic acid, succinic acid, acetic acid, benzoic acid, oxalic acid, citric acid, tartaric acid, carbonic acid or phosphoric acid. Furthermore, where the compounds of the disclosure carry an acidic moiety, suitable pharmaceutically acceptable salts thereof may include alkali metal salts, e.g. sodium or potassium salts, alkaline earth metal salts, e.g. calcium or magnesium salts; and salts formed with suitable organic ligands, e.g. quaternary ammonium salts.

Neutral forms of the compounds may be regenerated by contacting the salt with a base or acid and isolating the parent compound in a conventional manner. The parent form of the compound differs from the various salt forms in certain physical properties, such as solubility in polar solvents, but otherwise the salts are equivalent to the parent form of the compound for the purposes of this disclosure.

In addition to salt forms, the present disclosure may also provide compounds that are in a prodrug form. Prodrugs of the compounds are those compounds that readily undergo chemical changes under physiological conditions to provide the compounds. Prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Compounds can be an enantiomerically enriched isomer of a stereoisomer described herein. Enantiomer, as used herein, refers to either of a pair of chemical compounds whose molecular structures have a mirror-image relationship to each other. For example, a compound may have an enantiomeric excess of at least about 10%, 15%, 20%, 25%, 30%, 35%, 40%, 45%, 50%, 55%, 60%, 65%, 70%, 75%, 80%, 85%, 90%, 95%, 96%, 97%, 98%, or 99%.

A preparation of a compound may be enriched for an isomer of the compound having a selected stereochemistry, e.g., R or S, corresponding to a selected stereocenter. For example, the compound may have a purity corresponding to a compound having a selected stereochemistry of a selected stereocenter of at least about 60%, 65%, 70%, 75%, 80%, 85%, 90%, 95%, 96%, 97%, 98%, or 99%. A compound can, for example, include a preparation of a compound disclosed herein that is enriched for a structure or structures having a selected stereochemistry, e.g., R or S, at a selected stereocenter. “Substantially enantiomerically pure” refers to 95% or more enrichment in an indicated enantiomer.

A preparation of a compound may be enriched for isomers (subject isomers) which are diastereomers of the compound. Diastereomer, as used herein, refers to a stereoisomer of a compound having two or more chiral centers that is not a mirror image of another stereoisomer of the same compound. For example, the compound may have a purity corresponding to a compound having a selected diastereomer of at least about 60%, 65%, 70%, 75%, 80%, 85%, 90%, 95%, 96%, 97%, 98%, or 99%.

When no specific indication is made of the configuration at a given stereocenter in a compound, any one of the configurations or a mixture of configurations is intended.

Compounds may be prepared in racemic form or as individual enantiomers or diastereomers by either stereospecific synthesis or by resolution. The compounds may, for example, be resolved into their component enantiomers or diastereomers by standard techniques, such as the formation of stereoisomeric pairs by salt formation with an optically active base, followed by fractional crystallization and regeneration of the free acid. The compounds may also be resolved by formation of stereoisomeric esters or amides, followed by chromatographic separation and removal of the chiral auxiliary. Alternatively, the compounds may be resolved using a chiral HPLC column. The enantiomers also may be obtained from kinetic resolution of the racemate of corresponding esters using lipase enzymes.

### Synthesis of Compounds

Compounds may be synthesized from commercially available starting materials. Exemplary syntheses are illustrated below in the Examples.

Other methods of synthesizing the compounds of the formulae herein will be evident to those of ordinary skill in the art. Synthetic chemistry transformations and protecting group methodologies (protection and deprotection) useful in synthesizing the compounds are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

### Evaluation of Compounds

Compounds may be analyzed using a number of methods, including ex vivo and in vivo methods.

For example, the GABA_{A} subunit selectivity of compounds can be evaluated using competitive binding assays. Such assays have been previously described (Choudhary et al. Mol Pharmacol. 1992, 42, 627-33; Savić et al. Progress in Neuro-Psychopharmacology & Biological Psychiatry, 2010, 34, 376-386). The assays involve the use of a radiolabeled compound known to bind to GABA_{A} receptors, such as [³H]flunitrazepam. Membrane proteins can be harvested and incubated with the radiolabeled compound, and non-specific binding can be evaluated by comparing binding of the radiolabeled compound to another, non-labeled compound (e.g., diazepam). Bound radioactivity can be quantified by liquid scintillation counting. Membrane protein concentrations can be determined using commercially available assay kits (e.g., from Bio-Rad, Hercules, CA).

Compounds can also be evaluated in electrophysiological assays in Xenopus oocytes, or in transfected cell lines. Compounds can be pre-applied before the addition of GABA, which can then be co-applied with the compounds until a peak response is observed. Between applications, oocytes or transfected cell lines can be washed to ensure full recovery from desensitization. For current measurements of the GABA response magnitude (Fisher et al. Mol Pharmacol, 1997, 52, 714-724), the oocytes or transfected cells can be impaled with microelectrodes, and recordings performed using voltage clamps.

Compounds described herein may be GABA_{A} receptor ligands which exhibit antidepressant, anxiolytic or pro-cognitive activities due to increased agonist efficacy at GABA_{A}/α5 and/or GABA_{A}/α2, GABA_{A}/α3 or GABA_{A}/α_{2/3} receptors. The compounds may possess at least 2-fold, suitably at least 5-fold, and advantageously at least a 10-fold, selective efficacy for GABA_{A}/α5 receptors relative to the GABA_{A}/α1 receptors. However, compounds which are not selective in terms of their agonist efficacy for the GABA_{A}/α5 receptors are also encompassed within the scope of the present disclosure. Such compounds will desirably exhibit functional selectivity by demonstrating pro-cognitive and/or antidepressant activity with decreased sedative-hypnotic/muscle relaxant/ataxic activity due to decreased efficacy at GABA_{A}/α1 receptors.

GABAergic receptor subtype selective compounds which are ligands of the GABAA receptors acting as agonists or partial agonists or positive allosteric modulator (PAM) are referred to hereinafter as “GABA_{A} receptor agonists” or “GABA_{A} receptor partial agonists” or “agonists” or “partial agonists” or “PAM”. In particular these are compounds that are ligands of the benzodiazepine (BZ) binding site of the GABA_{A} receptors, and hence acting as BZ site agonists, partial agonists or PAMs. Such ligands also include compounds acting at the GABA site or at modulatory sites other than the benzodiazepine site of GABA_{A} receptors.

GABAergic receptor subtype selective compounds act as agonists, partial agonists or PAM by selectively or preferentially activating the GABA_{A}/α5 receptors, as compared to the GABA_{A}/α₁ receptors. A selective or preferential therapeutic agent has less binding affinity or efficacy to the GABA_{A}/α₁ receptors compared to the GABA_{A}/α₅, GABA_{A}/α₂ or GABA_{A}/α₃ receptors. Alternatively, the agent binds with a comparable affinity to GABA_{A}/α₅, GABA_{A}/α₁, GABA_{A}/α₂, and GABA_{A}/α₃ receptors but exerts preferential efficacy of the GABA_{A}/α₅ receptor activation compared to the GABA_{A}/α₁ receptors. Alternatively, a selective agent can also have greater or lesser binding affinity to GABA_{A}/α₂ and GABA_{A}/α₃ receptors relative to GABA_{A}/α₅. The Bz/GABA agonists act at the benzodiazepine site of the respective GABA_{A} receptors but are not restricted to this drug binding domain in its receptor interactions.

Pharmacokinetic properties of the GABA receptor subtype specific compounds are assessed measuring compound content in plasma and brain (Piantadosi et al. Front Pharmacol, 2016, 7, 446) following intra peritoneal, per esophagus and intravenous administration. Mice and rats receive the different compounds at different doses. Serial brain and plasma concentrations are obtained at different times and LC/MS/MS is used to measure the concentration of each compound in each sample, Using this assay, the Cₘₐₓ, Tₘₐₓ, AUC₀₋₁₂, AUC_{0-∞}, T_{1/2} and β are measured for each compound in each tissue, each route of administration for both species. Compounds with optimal pharmacokinetic properties are considered for potential therapeutic intervention.

Metabolic availability of the GABA receptor subtype specific compounds are assessed by measuring the rate of degradation of the compound using human and mouse liver microsomal assay. Liver microsomes are incubated with each compound and half-life, intrinsic clearance and metabolic rates are measured. Compounds with optimal metabolic parameters are considered for potential therapeutic intervention.

Cell toxicity and viability can be evaluated in in vitro conditions. Human cells (HEPG2 and/or HEK293 cell lines from liver and kidney, respectively) are incubated with the compounds and the cell viability assay is performed to determine toxicity of the compounds. Compounds with a LD₅₀ superior to 100 μM are considered to have no toxic effect on cell viability.

Other methods for evaluating compounds are known to those skilled in the art. For evaluating potential to improve cognitive functions, numerous behavioral tests exist in rodents and other mammals, including but not limited to tests for attention, visual and auditory processing, motor function, short term and working memory, learning and memory processes for multiple cognitive dimensions (procedural, declarative, spatial/reference memory etc.).

Declarative and procedural cognitive functions can be evaluated using various mazes, including the Morris water maze, the radial maze, the Barnes' hole board etc (Gallagher et al, Behav Neurosci, 1993, 107,618-626; Vorhees and Williams, ILAR J, 2014, 55,310-332; Schwabe et al. Neurosci & Biobehavioral review, 2010, 34, 584-591). These paradigms test learning and memory abilities, and are used to identify efficacy of compounds in improving learning and memory capacities of animals to refer to their environment to solve a challenge. Animals are trained to learn the task and to recall the information during a retrieval/probe test. Methods in the field for testing potential pro-cognitive properties of compounds include administration of the compounds during the learning and/or the recall phases of the tests. Compounds that reduce the latency to succeed and/or the number of errors in the task during the learning and/or the recall phases are considered to have potential pro-cognitive actions.

Other tests measuring cognitive functions include discrimination task between familiar and novel objects/subjects. These tests include but are not limited to variation of the novel object recognition test, place recognition test, the social novelty discrimination, three chamber test, etc. (Morici et al, Behav Brain Research, 2015, 292, 241-251; Millan and Bales, Neurosci & Biobehavioral Reviews, 2013, 37, 2166-2180). The concept of these tests is to assess the ability of the animal to discriminate between a familiar and a novel element in its environment. Compounds that increase the time interacting with the novel element are considered to have pro-cognitive properties.

Short-term and working memory functions can also be assessed using different tasks employing Morris water maze, Y-Maze or T-Maze apparatuses or variations (Lalonde, Neurosci & Biobehavioral Reviews, 2002, 26, 91-104). Briefly, these tests are based on the ability of the animals to keep information for a short period of time, in order to recall the information shortly after the acquisition. Efficient processes of working memory require irrelevant information to be “erased” to allow the acquisition of new relevant information. For example, short-term working memory can be assessed by exploiting the innate tendency of the animals to explore a new environment and rely on their propensity to spontaneously alternate when repetitively given the choice to visit two different arms of the apparatus. Following multiple trials, the number of errors can increase because of a load of interference. The load of interference can be modulated by variation of the delay between trials, with long inter-trial intervals increasing the load of interference indexed as an increase in the number of errors. Stress paradigms, normal aging or other models of neuropsychiatric or neurodegenerative disorders can be used to reduce cognitive performance in these tests. This would be characterized by an increase in the number of errors (i.e. increasing errors in water maze tasks, or a performance around the chance level of 50% in alternation tasks). Compounds that reverse these deficits are considered to have pro-cognitive actions.

For evaluating antidepressant potential of compounds, numerous tests exist in rodents and other mammals to assess emotionality, including but not limited to tests for anxiety-, behavioral despair-, helplessness- or anhedonia-like behavioral responses.

Per standard methods in the field, forced swim test and tail suspension test (Castagne et al. Curr Protoc Neurosci, 2011, Chapter 8) are predominantly used to screen antidepressant action in rodents but are also employed as behavioral measures of despair in response to stress. Briefly rodents are acutely or sub-chronically injected for testing potential antidepressant compounds. Rodents are placed in an inescapable situation (in a tank of water or hanged by the tail) and a count of the immobile time is measured as an index of resignation to a state of despair. Compounds that reduced immobility in these tests are considered to have potential antidepressant actions.

Other methods of evaluating antidepressant actions include behavioral reactivity to aversive stimuli such as learned helplessness test or fear conditioning (Cryan and Mombereau, Molecular Psychiatry, 2004, 9, 326; Phillips and LeDoux, Behavioral Neuroscience, 1992, 106, 274-285). Both tests rely on the ability of the animals to cope to an adverse conditioned and non-conditioned stimulus. Foot shocks are applied to the animals and the behavioral response produced is used as an index for potential antidepressant properties of the compounds. Compounds that decrease the number and/or latency to escape the foot shock in the learned helplessness test, as well as the time spent freezing in the fear conditioning paradigm are considered to have potential antidepressant actions.

Potential antidepressant activity of the compounds can also be evaluated for their properties to reverse anhedonia-like behaviors observed in animal models exhibiting altered emotionality. For example, the novelty induced hyperphagia, the sucrose preference or consumption, or the cookie tests can be employed to assess anhedonia in rodent. Anhedonia is characterized by a decrease in seeking pleasant experiences including but not limited to palatable solution or food (Willner, Neuropsychobiology, 2005, 52, 90-110; Nollet et al. Curr Protoc Neurosci, 2013, Chapter 5). Compounds that increase the seeking of pleasurable experience are considered to have potential antidepressant actions.

Other methods for evaluating compounds are known to those skilled in the art. Anxiety symptoms are often co-morbid and difficult to separate from the mood spectrum observed in depression or AD. For example, an assessment of anxiolytic effects of compounds can be accomplished objectively and quantitatively with operant-based conflict procedures, as described in Fischer et al. Neuropharmacology 59 (2010) 612-618. Briefly, behavior which is positively reinforced can be suppressed in these procedures by response-contingent administration of a noxious stimulus such as mild electric shock. If a compound has an anxiolytic effect it increases the rates of responding that are normally suppressed by response-contingent delivery of shock. The strength of conflict procedures is their predictive validity with respect to expected therapeutic effects in humans.

Potential anxiolytic activity and locomotor activity can be evaluated in a home-cage like apparatus. Animals with high level of anxiety have a propensity to spend more time hidden in their shelter, limiting their exploratory behaviors. The same setting can be used with the application of a light challenge. Anxious animals continue avoiding the lit zone even after the end of the light challenge (Pham et al. J Neurosci Methods, 2009, 178, 323-326). Compounds that increase the exploration and limit this avoidance behavior in these type of test are considered to have potential antidepressant/anxiolytic actions.

Anxiolytic activity can also be evaluated in the light/dark box test by a method developed by Crawley (Neurosci Biobehav Rev 1985, 9, 37-44). The light/dark box is a simple noninvasive test for anxiolytic activity. Compounds that increased the latency to enter the dark box and/or increase the time spent in the lit box are considered to have an anxiolytic action.

Potential anxiolytic activity can be measured in the elevated plus maze and the open-field tests (Bailey and Crawley, Methods of Behavior Analysis in Neuroscience, 2009, 2^{nd} Edition), In both tests, a conflict is created between the animal's natural tendency to explore and its innate fear of predator threat in an exposed environment. Rodents are placed in the center of the maze/field under a bright light condition. The number of entries as well as the time spent in the exposed areas (open arms/center of the arena) are recorded. Compounds that increase these parameters without decreasing the exploratory behavior are considered to have anxiolytic actions.

Other methods to assess anxiolytic properties of compounds may employ tests that rely on the conflict between the drive for food or sweetened solution and the fear of being placed in a novel environment. In these tests, the latency to approach and to feed in the novelty suppressed feeding or the cookie tests as well as the latency to drink the condensed milk in the novelty induced hyperphagia are outcome measures (Nollet et al. Curr Protoc Neurosci, 2013, Chapter 5). Compounds that decrease theses parameters are considered to have potential anxiolytic properties.

The marble burying assay (Deacon, Nat Protocols, 2006, 1, 122: Kinsey et al., Pharmacol Biochem Behav 2011, 98, 21) is another anxiolytic test. Mice or rats are placed in a cage with marbles on top of bedding material which they can dig to bury the marbles. The rodents are then timed and the number of marbles buried is counted. A reduction in marble burying compared to control is considered an anxiolytic effect.

Cognition, depression, anxiety and other behavioral dimensions associated with disorders can be evaluated under normal baseline conditions or using rodent models of the conditions or symptoms. Examples of such models include the unpredictable chronic mild stress, the chronic restraint stress and the social defeat paradigm. For instance, “impaired cognition” and “depressive-like” states can be induced in rodents using a prolonged protocol of unpredictable mild stressors over several weeks. Mild stressors are typically but not exclusively applied in a random manner to provide an unpredictable environment. Mild stressors may include but not limited to changes in light cycles, changes in cage bedding, switching cages, exposure to predator odor, to noise or bright light, social stressor, exposure to aggressive mice. Rodents exposed to these paradigms typically display altered cognitive functions and increased depression- and anxiety-related behaviors and can be assessed by various behavioral tests including but not limited to the ones described herein. Compounds that reverse deficits in these tests can be considered for therapeutic indication.

Cognition, depression, anxiety, and other behavioral dimensions associated with these disorders can be evaluated in rodent models where genetic engineering has been used to induce a cellular or molecular pathology that causes behavioral or physiological changes associated with the disorders. For example, the acute inhibition of SST GABA neurons using a chemogenetic approach induces elevated behavioral emotionality (Sournier and Sibille, Neuropsychopharmacology, 2014, 39:9, 2252-62), furthermore, compounds that reduce emotionality can be considered for therapeutic indication.

Deficits in cognitive function associated with neurodegenerative disorders can also be evaluated in the multiple rodent models that have been developed using genetic manipulations to induce pathologies observed in the human brain, such as increased beta-amyloid plaques and/or neurofibrillary tangles. For instance, the TgCRND8 murine model of AD expresses a mutant human bAPP transgene TgCRND8 mice and displays spatial learning deficits at 3 months of age that are accompanied by both increasing levels of Amyloid beta plaques and neurofibrillary tangles in the brain (Janus et al, Nature, 2000, 408:979-982). Data suggests that compounds that reverse or improve cognitive symptoms in these models would be effective in normal or disease related loss of cognition or pathological conditions.

For evaluation of potential to treat schizophrenia, compounds may be tested using a mouse model as described in Gill et al. Neuropsychopharrmacology 2011, 36: 1903-1911. This mouse model of schizophrenia arises from a development disturbance induced by the administration of a DNA-methylating agent, methylazoxymethanol acetate (MAM), to pregnant dams on gestational day 17. The MAM-treated offspring display structural and behavioral abnormalities, consistent with those observed in human patients with schizophrenia. Antagonism or genetic deletion of the α5GABA_{A} receptor (α5GABA_{A} R) leads to behaviors that resemble some of the behavioral abnormalities seen in schizophrenia, including prepulse inhibition to startle and impaired latent inhibition. The MAM model can be used to show the effectiveness of a benzodiazepine-positive allosteric modulator (PAM) compound selective for the α5 subunit of the GABA_{A}R. In Gill et al., the pathological increase in tonic dopamine transmission in the brain was reversed, and behavioral sensitivity to psychostimulants observed in MAM rats was reduced. The data suggests that such compounds would be effective in alleviating dopamine-mediated psychosis.

Measures of the global locomotion can also be used to assess potential sedative effect of the tested compounds. The mouse is placed in a home-cage like arena and distance travelled is monitored for 30-60 minutes (Tang et al. Behavioral Brain Research, 2002, 136, 555-569). Compounds that induce drastic reduction in the distance travelled are considered having sedative or undesirable side effects.

Measures of spatial and motor coordination can also be assessed to assess the sedative-hypnotic/muscle relaxant/ataxic activity of compounds. The sensorimotor rotarod test is typically used for these assessments (Voss et al. European Journal of Pharmacology, 2003, 482, 215-222). This test is carried out 10, 30 and 60 minutes after injection of the test compound. Mice or rats are tested on a rotating rod (rotarod) at 15 rpm for maximum 3 min and the time of fall is recorded. Falling before the 3 minutes period of testing would be indicative of any locomotor coordination impairment induced by the compound.

Such compounds will desirably exhibit functional selectivity by demonstrating pro-cognitive and/or antidepressant activity with decreased sedative-hypnotic/muscle relaxant/ataxic activity due to decreased efficacy at GABA_{A}/α1 receptors.

Compounds activating GABA-A receptors as well as compounds selective for GABA_{A} receptor subunits often display anti-epileptic activity, due to their general suppression of neural activity. Accordingly anti-epileptic properties of the compounds can be tested for the ability to suppress seizures in several standard rat and mouse models of epilepsy, as described in U.S. Patent Application Publication No. US 2011/0261711. Anticonvulsant activity of compounds can be compared to diazepam. The standard models incorporated into anticonvulsant screening include the maximal electroshock test (MES), the subcutaneous Metrazol test (scMet), and evaluations of toxicity (TOX). The data for each condition can be presented as a ratio of either the number of animals protected or toxic (loss of locomotor activity) over the number of animals tested at a given time point and dose.

The MES is a model for generalized tonic-clonic seizures and provides an indication of a compound's ability to prevent seizure spread when all neuronal circuits in the brain are maximally active. These seizures are highly reproducible and are electrophysiologically consistent with human seizures. For all tests based on MES convulsions, 60 Hz of alternating current (50 mA in mice, 150 in rats) is delivered for by corneal electrodes which have been primed with an electrolyte solution containing an anesthetic agent (0.5% tetracaine HCL). For Test 1, mice are tested at various intervals following doses of 30, 100 and 300 mg/kg of test compound given by ip injection of a volume of 0.01 mL/g. In Test 2, rats are tested after a dose of 30 mg/kg (po) in a volume of 0.04 mL/g. Test 3 uses varying doses administered via i.p. injection, again in a volume of 0.04 ml/g. An animal is considered “protected” from MES-induced seizures upon abolition of the hindlimb tonic extensor component of the seizure (Swinyard, E. A., et al. in Antiepileptic Drugs, Levy, R. H. M., et al., Eds.; Raven Press: New York, 1989; pp 85-102; White, H. S. et al., Ital J Neurol Sci. 1995a, 16, 73-7; White, H. S., et al., in Antiepileptic Drugs, Levy, R. H. M., Meldrum, B. S., Eds.; Raven Press: New York, pp 99-110, 1995b).

Subcutaneous injection of the convulsant Metrazol produces clonic seizures in laboratory animals. The scMet test detects the ability of a test compound to raise the seizure threshold of an animal and thus protect it from exhibiting a clonic seizure. Animals can pretreated with various doses of the test compound (in a similar manner to the MES test, although a dose of 50 mg/kg (po) is the standard for Test 2 scMet). At the previously determined TPE of the test compound, the dose of Metrazol which will induce convulsions in 97% of animals (CD.sub.97: 85 mg/kg mice) is injected into a loose fold of skin in the midline of the neck. The animals can be placed in isolation cages to minimize stress (Swinyard et al. J. Physiol. 1961, 132, 97-0.102) and observed for the next 30 minutes for the presence or absence of a seizure. An episode of clonic spasms, approximately 3-5 seconds, of the fore and/or hindlimbs, jaws, or vibrissae is taken as the endpoint. Animals which do not meet this criterion are considered protected.

To further characterize the anticonvulsant activity of compounds, a hippocampus kindling screen can be performed. This screen is a useful adjunct to the traditional MES and scMet tests for identification of a substance potential utility for treating complex partial seizures.

Benzodiazepines can be highly effective drugs in certain treatment paradigms. They are routinely employed for emergency situations such as status epilepticus and other acute conditions. But their use in chronic convulsant diseases has been limited due to side effects such as sedation and with high doses respiratory depression, hypotension and other effects. Further it has long been purported that chronic administration of this class of drugs can lead to tolerance to the anticonvulsant effects. This has limited their utility as first line treatment for chronic anticonvulsant conditions. Discovery of a potent BDZ with a decreased side effect profile and efficacy over extended treatment periods would be highly desirable.

### Compositions and Routes of Administration

In another aspect, the disclosure provides pharmaceutical compositions comprising one or more compounds of this disclosure in association with a pharmaceutically acceptable carrier. Such compositions may be in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. It is also envisioned that compounds may be incorporated into transdermal patches designed to deliver the appropriate amount of the drug in a continuous fashion. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture for a compound of the present disclosure, or a pharmaceutically acceptable salt thereof. When referring to these preformulation compositions as homogeneous, it is meant that the active ingredient is dispersed evenly throughout the composition so that the composition may be easily subdivided into equally effective unit dosage forms such as tablets, pills and capsules. This solid preformulation composition is then subdivided into unit dosage forms of the type described above containing from 0.1 to about 500 mg of the active ingredient of the present disclosure. Typical unit dosage forms contain from 1 to 100 mg, for example, 1, 2, 5, 10, 25, 50, or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compositions of the present disclosure may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

Suitable dosage level is about 0.01 to 250 mg/kg per day, about 0.05 to 100 mg/kg per day, or about 0.05 to 5 mg/kg per day. The compounds may be administered on a regimen of 1 to 4 times per day, or on a continuous basis via, for example, the use of a transdermal patch.

Pharmaceutical compositions for enteral administration, such as nasal, buccal, rectal or, especially, oral administration, and for parenteral administration, such as intravenous, intramuscular, subcutaneous, peridural, epidural or intrathecal administration, are suitable. The pharmaceutical compositions comprise from approximately 1% to approximately 95% active ingredient, or from approximately 20% to approximately 90% active ingredient.

For parenteral administration including intracoronary, intracerebrovascular, or peripheral vascular injection/infusion preference is given to the use of solutions of the subunit selective GABA_{A} receptor agonist, and also suspensions or dispersions, especially isotonic aqueous solutions, dispersions or suspensions which, for example, can be made up shortly before use. The pharmaceutical compositions may be sterilized and/or may comprise excipients, for example preservatives, stabilizers, wetting agents and/or emulsifiers, solubilizers, viscosity-increasing agents, salts for regulating osmotic pressure and/or buffers and are prepared in a manner known per se, for example by means of conventional dissolving and lyophilizing processes.

For oral pharmaceutical preparations suitable carriers are especially fillers, such as sugars, for example lactose, saccharose, mannitol or sorbitol, cellulose preparations and/or calcium phosphates, and also binders, such as starches, cellulose derivatives and/or polyvinylpyrrolidone, and/or, if desired, disintegrators, flow conditioners and lubricants, for example stearic acid or salts thereof and/or polyethylene glycol. Tablet cores can be provided with suitable, optionally enteric, coatings. Dyes or pigments may be added to the tablets or tablet coatings, for example for identification purposes or to indicate different doses of active ingredient. Pharmaceutical compositions for oral administration also include hard capsules consisting of gelatin, and also soft, sealed capsules consisting of gelatin and a plasticizer, such as glycerol or sorbitol. The capsules may contain the active ingredient in the form of granules, or dissolved or suspended in suitable liquid excipients, such as in oils.

Transdermal application is also considered, for example using a transdermal patch, which allows administration over an extended period of time, e.g. from one to twenty days.

### Methods of Treatment

Further provided herein are methods of treating a disorder or condition with the compounds disclosed herein. Further provided are methods of cognitive and mood remediation in neurological disorders.

The disorder may be selected from Alzheimer's Disease (AD), dementia, Traumatic Brain Injury (TBI), cerebrovascular disease, anesthesia, post-traumatic stress disorder (PTSD), depression such as major depression and persistent depressive disorder and bipolar depression, schizophrenia, alcoholism, addiction, anxiety disorder, epilepsy, neuropathic pain, autism spectrum disorder, or a combination thereof.

Anxiety disorder is divided into generalized anxiety disorder, phobic disorder, and panic disorder; each has its own characteristics and symptoms and they require different treatment. Particular examples of anxiety disorders include generalized anxiety disorder, panic disorder, phobias such as agoraphobia, social anxiety disorder, obsessive-compulsive disorder, post-traumatic stress disorder, separation anxiety and childhood anxiety disorders.

There are many different epilepsy syndromes, each presenting with its own unique combination of seizure type, typical age of onset, EEC findings, treatment, and prognosis. Exemplary epilepsy syndromes include, e.g., Benign centrotemporal lobe epilepsy of childhood, Benign occipital epilepsy of childhood (BOEC), Autosomal dominant nocturnal frontal lobe epilepsy (ADNFLE), Primary reading epilepsy, Childhood absence epilepsy (CEA), Juvenile absence epilepsy, Juvenile myoclonic epilepsy (JME), Symptomatic localization-related epilepsies, Temporal lobe epilepsy (TLE), Frontal lobe epilepsy, Rasmussen's encephalitis, West syndrome, Dravet's syndrome, Progressive myoclonic epilepsies, and Lennox-Gastaut syndrome (LGS). Genetic, congenital, and developmental conditions are often associated with epilepsy among younger patients. Tumors might be a cause for patients over age 40. Head trauma and central nervous system infections may cause epilepsy at any age.

Schizophrenia is a mental disorder characterized by a breakdown of thought processes and by poor emotional responsiveness. Particular types of schizophrenia include paranoid type, disorganized type, catatonic type, undifferentiated type, residual type, post-schizophrenic depression and simple schizophrenia.

Autism spectrum disorder encompasses a range of phenotypes expressed during neurodevelopment, characterized by persistent deficits in social communication and interaction across various contexts.

The treatment can also be directed at a symptom dimension (e.g. cognition, mood) across disorders, from neuropsychiatric (e.g., depression, schizophrenia) to neurodegenerative (e.g. AD) disorders, consistent with a dimensional perspective of underlying brain pathologies.

The methods may include administering to a subject in need thereof a compound or composition as described herein.

The following non-limiting examples are intended to be purely illustrative of some aspects and embodiments, and show specific experiments that were carried out in accordance with the disclosure.

### Examples

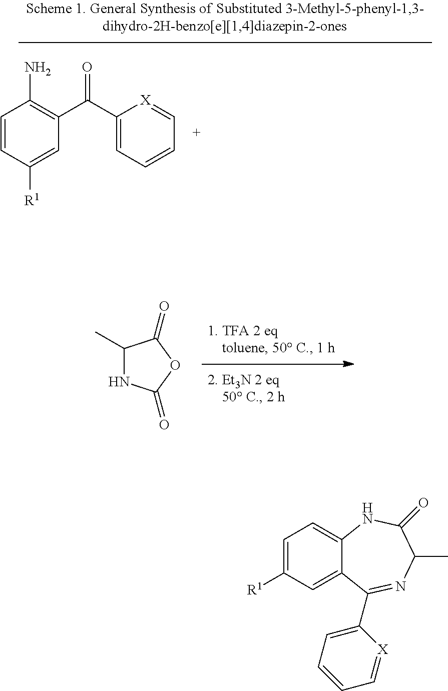

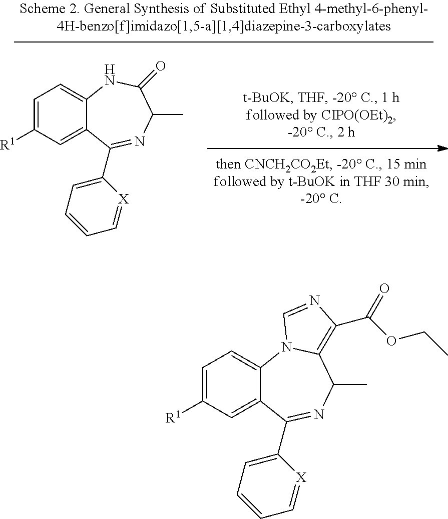

In Schemes 1 and 2, R¹ may be phenyl, chloro, bromo, ethynyl, or cyclopropyl and X may be N, C—Cl, or C—F. The ethyl esters in Scheme 2 may be converted to the corresponding carboxylic acid using well-known methods, such as hydrolysis under basic conditions.

### General Procedure for Other Amides

A mixture of a carboxylic acid (1 equivalent), thionyl chloride (10 equivalents) and dry dichloromethane is put into an oven dried round bottom flask under argon. This suspension is allowed to reflux at 60° C. for 2 hours under argon. The organic solvent and excess thionyl chloride are evaporated under reduced pressure which is repeated 5 times with dry dichloromethane. The product obtained is dissolved in dry dichloromethane and cooled to 0° C. for 10 min under argon. Then an appropriate amine (2 equivalents), followed by Et₃N (1 equivalent) is added to the reaction mixture at 0° C. and the mixture is then allowed to warm to room temperature and stirred for approximately 7 hours. After the completion of the reaction, the solvent is removed under reduced pressure. The product is treated with ice-cold water and extracted with dichloromethane. The combined organic layers are washed with brine. The solvent is removed under reduced pressure and the product is purified by column chromatography to yield the corresponding pure amides.

### (S)-8-ethynyl-N,4-dimethyl-6-(pyridin-2-yl)-4H-benzo[f]imidazo[1,5-a][1,4]diazepine-3-carboxamide (MYM-III-29)

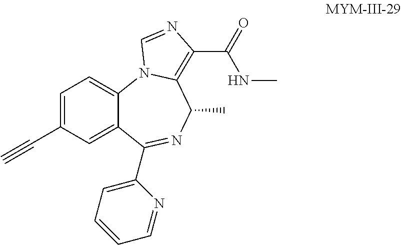 [SMILES: C#Cc1ccc2c(c1)C(c1ccccn1)=N[C@@H](C)c1c(C(=O)NC)ncn1-2]

The starting ester, ethyl (S)-8-ethynyl-4-methyl-6-(pyridin-2-yl)-4H-benzo[f]imidazo[1,5-a][1,4]diazepine-3-carboxylate (1 g, 2.69 mmol) was charged in a sealed vessel fitted with a septum at −30° C. and then methyl amine (20 mL, 33% wt solution in EtOH) was added. The vessel was sealed with a screw-cap and stirred at 60° C. for 12 hours. The solution was then cooled to room temperature and the methyl amine and ethanol were removed under reduced pressure. The resulting residue was purified by flash column chromatography (silica gel, EtOAc) to afford the pure title compound as off-white powder (063 g, 65%): ¹H NMR (500 MHz, CDCl3) δ 8.56 (d, J=4.6 Hz, 1H), 8.06 (d, J=7.9 Hz, 1H), 7.82 (t, J=7.7 Hz, 1H), 7.76 (s, 1H), 7.70 (d, J=8.3 Hz, 1H), 7.52 (d, J=8.3 Hz, 1H), 7.47 (s, 1H), 7.35 (dd, J=6.7, 5.5 Hz, 1H), 7.17 (s, 1H), 6.90 (q, J=7.2 Hz, 1H), 3.15 (s, 1H), 2.97 (s, 3H), 1.28 (d, J=7.2 Hz, 3H). IC NMR (126 MHz, CDCl3) δ 164.69 (s), 163.40 (s), 157.72 (s), 148.38 (s), 138.74 (s), 136.83 (s), 136.08 (s), 135.95 (s), 134.84 (s), 133.63 (s), 131.57 (s), 128.01 (s), 12445 (s), 124.07 (s), 12225 (s), 120.70 (s), 81.84 (s), 79.18 (s), 49.63 (s), 25.57 (s), 14.74 (s). HRMS (ESI/IT-TOF) m/z: [M+H]+Calcd for C₂₁ H₁₇N₅O 356.1506; found 356.1474. % EE=>99% (HPLC). % Purity=>99% (HPLC).

### (R)-6-(2-fluorophenyl)-N,N,4-trimethyl-8-phenyl-4H-benzo[f]imidazo[1,5-a][1,4]diazepine-3-carboxamide (MYM-III-41)

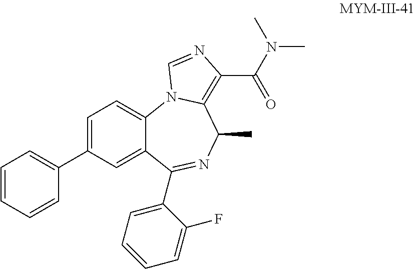 [SMILES: C[C@H]1N=C(c2ccccc2F)c2cc(-c3ccccc3)ccc2-n2cnc(C(=O)N(C)C)c21]

A mixture of (R)-6-(2-fluorophenyl)-4-methyl-8-phenyl-4H-benzo[f]imidazo[1,5-a][1,4]diazepine-3-carboxylic acid (MYM-III-37) (350 mg, 0.85 mmol), thionyl chloride (0.6 mL, 8.5 mmol), a catalytic amount of anhydrous N,N-dimethyl formamide (0.07 mL, 0.085 mmol) and dry dichloromethane (20 mL) were charged in an oven dried round bottom flask under argon. This suspension, which formed, was allowed to reflux at 50° C. for 1 hour under argon. The organic solvent and excess thionyl chloride were removed under reduced pressure on a rotary evaporator and it was repeated five times with anhydrous dichloromethane (5×10 mL). The resulting yellow residue in anhydrous dichloromethane (20 mL) and cooled to 0° C. for 10 minutes under argon followed by addition of N,N-dimethylamine solution (4.25 mL, 8.5 mmol). The mixture was then allowed to warm to room temperature and stirred for an hour. After the completion of the reaction, the mixture was diluted with ice cold water (15 mL) and extracted with dichloromethane (3×20 mL). The combined organic layers were washed with brine (20 mL), dried (Na₂SO₄) and the residue was purified with silica gel flash chromatography (EtOAc and 1% trimethylamine) to get pale yellow solid of MYM-III-41 (0.29 g, 80%). ¹H NMR (500 MHz, CDCl3) δ 7.98 (s, 1H), 7.83 (d, J=8.0 Hz, 1H), 7.67 (td, J=7.5, 1.5 Hz, 1H), 7.62 (d, J=8.2 Hz, 1H), 7.52-7.47 (in, J=7.2 Hz, 3H), 7.45 (t, J=7.2 Hz, 3H), 7.41-7.37 (m, 1H), 7.25 (t, J=7.5 Hz, 1H), 7.02 (t, J=9.2 Hz, 1H), 4.40 (q, J=6.7 Hz, 1H), 3.14 (s, 3H), 3.03 (s, 3H), 1.96 (d, J=6.6 Hz, 3H). ¹³C NMR (126 MHz, CDCl3) δ 166.36 (s), 163.43 (s), 160.29 (d, J=251.4 Hz), 140.38 (s), 138.95 (s), 134.84 (s), 133.83 (s), 132.43 (s), 131.92 (d, J=7.9 Hz), 131.38 (s), 131.36 (s), 130.67 (s), 130.32 (s), 129.51 (s), 129.03 (s), 128.61 (s), 128.20 (s), 128.03 (d, J=12.4 Hz), 127.13 (s), 124.47 (d, J=2.5 Hz), 123.06 (s), 116.14 (d, J=21.4 Hz), 52.26 (s), 39.16 (s), 35.06 (s), 18.55 (s). HRMS (ESI/IT-TOF) m/z: [M+H]+Calcd for C₂₇H₂₃FN₄O 439.1929; found 439.1908. % EE=>99% (HPLC). % Purity=>99% (HPLC).

### (R)-8-Cyclopropyl-N-ethyl-6-(2-fluorophenyl)-4-methyl-4H-benzo[f]imidazo[1,5-a][1,4]diazepine-3-carboxamide (GL-III-68)

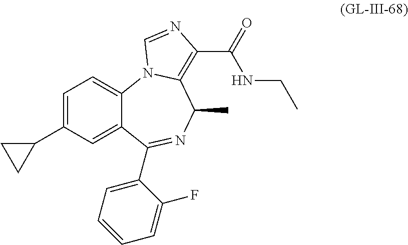 [SMILES: CCNC(=O)c1ncn2c1[C@@H](C)N=C(c1ccccc1F)c1cc(C3CC3)ccc1-2]

To a solution of (R)-8-bromo-N-ethyl-6-(2-fluorophenyl)-4-methyl-4H-benzo[f]imidazo[1,5-a][1,4]diazepine-3-carboxamide (3.0 g, 6.8 mmol) in toluene (50 mL) and water (1.46 mL), cyclopropyl boronic acid (3 g, 34.0 mmol), potassium phosphate (6.3 g, 29.7 mmol) and bis(triphenylphosphine)palladium(II) diacetate (1.52 g, 2.0 mmol) were added under argon. A reflux condenser was attached and the mixture was degassed under vacuum with argon. This process was repeated four times. The mixture was stirred and heated to 100° C. After 12 hours, the reaction was completed on analysis by TLC (silica gel) and it was then cooled to room temperature. Water (20 mL) was added and the mixture was extracted with EtOAc (3×25 mL), after which the filtrate was washed with brine (20 mL), dried (Na₂SO₄) and concentrated under reduced pressure. The black residue which resulted was purified by a wash column (silica gel, EtOAc/hexane 4:1) to afford the title compound as a white solid (2.3 g, 85%): mp 189-190° C.; 1H NMR (300 MHz, CDCl₃) δ 7.79 (s, 1H), 7.61 (t, J=6.6 Hz, 1H), 7.41 (t, J=10.0 Hz, 2H), 7.31-7.14 (m, 3H), 7.09-6.91 (m, 2H), 6.86 (d, J=7.0 Hz, 1H), 3.62-3.24 (m, 2H), 1.94-1.71 (m, 1H), 1.22 (dd, J=15.1, 7.6 Hz, 6H), 0.98 (d, J=7.9 Hz, 2H), 0.70-0.46 (m, 2H); ¹³C NMR (75 MHz, CDCl₃) δ 163.84 (s), 162.71 (s), 160.15 (d, J=249.0 Hz), 143.60 (s), 138.69 (s), 133.31 (s), 132.27 (s), 131.57 (s), 131.40 (s), 129.35 (s), 129.29 (s), 128.49 (s), 128.29 (s), 127.85 (s), 124.28 (s), 121.79 (s), 115.89 (d, J=21.8 Hz), 49.82 (s), 33.65 (s), 15.03 (s), 14.82 (s), 9.82 (s); HRMS (ESI/IT-TOF) m/z: [M+H] Calcd for C₂₄H₂₄FN₄O 403.1929; found 403.1927.

### (R)-6-(2-chlorophenyl)-8-ethynyl-N,N,4-trimethyl-4H-benzo[f]imidazo[1,5-a][1,4]diazepine-3-carboxamide (PM-II-26)

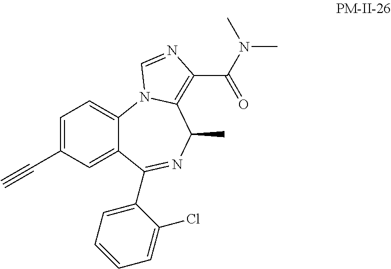 [SMILES: C#Cc1ccc2c(c1)C(c1ccccc1Cl)=N[C@H](C)c1c(C(=O)N(C)C)ncn1-2]

(R)-6-(2-Chlorophenyl)-8-ethynyl-4-methyl-4H-benzo[f]imidazo[1,5-a][1,4]diazepine-carboxylic acid (2 g, 5.3 mmol) was added into a flask of dry dichloromethane and resulted in a suspension. Thionyl chloride (7 equivalents) was added, and the solution was stirred at reflux for 2 hours. The reaction was complete after 2 hours as the hydrogen evolution stopped. The resulting acid chloride was dried under reduced pressure to remove excess thionyl chloride. The acid chloride was then dissolved in dry dichloromethane and dimethyl amine (6 equivalents) was added into an empty flask and the temperature was reduced to 0° C. with ice. Then the acid chloride was added dropwise to the amine over an hour and stirred for 3 hours at room temperature. The crude residue was purified by column chromatography (silica gel, ethyl acetate, 1% methanol) to yield pure PM-II-26 (1.5 g, 71%). ¹H NMR (500 MHz, CDCl₃): δ 7.91 (s, 1H), 7.71 (d, J=7.7 Hz, 1H), 7.57-7.49 (m, 2H), 7.40 (dd, J=63, 3.1 Hz, 2H), 7.34 (d, J=6.4 Hz, 1H), 7.30 (s, 1H), 4.37 (d, J=5.3 Hz, 1H), 3.15 (s, 1H), 3.13 (s, 2H), 2.98 (s, 2H), 1.92 (d, J=6.4 Hz, 3H). ¹³C NMR (126 MHz, CDCl3): δ 165.31 (s), 138.69 (s), 135.39 (s), 135.20 (s), 134.42 (s), 133.89 (s), 133.49 (s), 132.67 (s), 132.46 (s), 130.82 (s), 130.67 (s), 130.07 (s), 129.01 (s), 127.19 (s), 122.66 (s), 121.49 (s), 81.56 (s), 79.55 (s), 52.10 (s), 39.03 (s), 34.99 (s), 18.38 (s). HRMS (ESI/IT-TOF) m/z: [M+H]+Calcd for C23H19ClN4O 403.132; found 403.133.

### (S)-6-(2-chlorophenyl)-8-ethynyl-N,4-dimethyl-4H-benzo[f]imidazo[1,5-a][1,4]diazepine-3-carboxamide (FR-III-17)

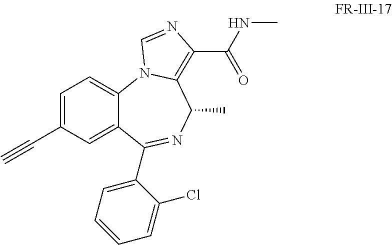 [SMILES: C#Cc1ccc2c(c1)C(c1ccccc1Cl)=N[C@@H](C)c1c(C(=O)NC)ncn1-2]

The starting ester, ethyl (S)-6-(2-chlorophenyl)-8-ethynyl-4-methyl-4H-benzo[f]imidazo[1,5-a][1,4]diazepine-3-carboxylate (1 g, 2.48 mmol) was charged a sealed vessel fitted with a septum at −30° C. and then methyl amine (15 mL, 33% wt solution in EtOH) was added. The vessel was sealed with a screw-cap and stirred at 60° C. for 12 hours. The solvents were evaporated and the crude residue was purified by column chromatography (silica gel, ethyl acetate, 1% methanol) to yield pure FR-III-17 (690 mg, 69%). ¹H NMR (500 MHz, CDCl3) δ 7.81 (s. 1H), 7.66 (d, J=7.3 Hz, 1H), 7.54 (d, J=8.3 Hz, 2H), 7.41-7.37 (m, 2H), 7.34 (s, 1H), 7.26 (s, 1H), 7.17 (s, 1H), 6.89 (q, 1H), 3.14 (s. 1H), 2.98 (s, 3H), 1.36 (d, J=4.3 Hz, 3H).

### General Procedure for Oxadiazoles

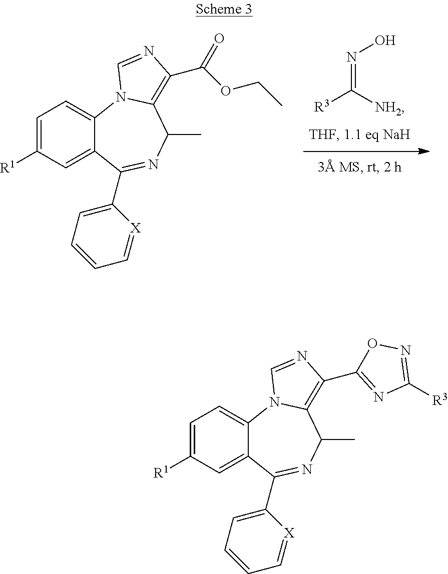

The ethyl esters (0.52 mmol) are dissolved in dry THF (20 mL) at room temperature under argon. In a separate flask which contained 3A molecular sieves, the corresponding amide oxime R³C(═NOH)NH₂ (2.08 mmol) is dissolved in dry THF (30 mL) under argon and treated with sodium hydride (60% dispersion in mineral oil, 0.57 mmol). The resulting mixture is stirred for 15 minutes, at which point the solution containing the ethyl ester is added. The resulting reaction mixture is stirred at room temperature for 2 hours until the starting material is consumed as indicated on analysis by TLC (silica gel). The reaction mixture is quenched with a saturated aqueous NaHCO₃ solution (50 mL). Water (50 mL) is then added and the product is extracted with EtOAc (3×100 mL). The organic layers are combined, washed with brine (30 mL) and dried (Na₂SO₄). The solvent is removed under reduced pressure. The resulting solid is purified by flash column chromatography (silica gel) to afford the oxadiazole.

In Scheme 3, R¹ may be phenyl, chloro, bromo, ethynyl, or cyclopropyl; X may be N, C—Cl, or C—F; and R³ may be an alkyl or cycloalkyl group.

### 5-(8-cyclopropyl-6-(2-fluorophenyl)-4H-benzo[f]imidazo[1,5-a][1,4]diazepin-3-yl)-3-ethyl-1,2,4-oxadiazole (MYM-III-43)

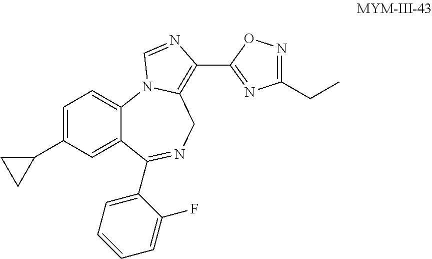 [SMILES: CCc1noc(-c2ncn3c2CN=C(c2ccccc2F)c2cc(C4CC4)ccc2-3)n1]

N′-Hydroxypropionimidamide (0.9 g, 10.2 mmol) was dissolved in dry THF (20 mL) under argon and treated with sodium hydride (60% dispersion in mineral oil, 0.068 g, 2.86 mmol) for an hour with molecular sieves, 3A in a round bottom flask. Ethyl 8-cyclopropyl-6-(2-fluorophenyl)-4H-benzo[f]imidazo[1,5-a][1,4]diazepine-3-carboxylate (1 g, 2.56 mmol) was dissolved in dry THF (20 mL) in a separate flask at room temperature under argon and then added to the flask containing N′-hydroxypropionimidamide. The reaction mixture, which resulted, was stirred at room temperature for 2 hours until the starting material was consumed as indicated on analysis by TLC (silica gel, EtOAc:Hex=50:50). The reaction mixture was quenched with aqueous NaHCO₃ solution (20 mL). Water (50 mL) was then added and the product was extracted with EtOAc (3×100 mL). The organic layers were combined, washed (aqueous 10% NaCl solution, 2×30 mL) and dried (Na₂SO₄). The solvent was removed under reduced pressure. The solid, which resulted, was purified by flash column chromatography (silica gel, EtOAc:Hex=50:50) to afford the pure oxadiazole, MYM-III-43 (white powder, 850 mg, 80%). ¹H NMR (500 MHz, CDCl3) δ 8.05 (s, 1H), 7.64 (t, J=7.5 Hz, 1H), 7.53 (d, J=8.3 Hz, 1H), 7.47-7.41 (m, 1H), 7.32 (d, J=8.3 Hz, 1H), 7.24 (t, J=7.5 Hz, 1H), 7.07 (s, 1H), 7.03 (t, J=9.4 Hz, 1H), 6.14 (d, J=10.9 Hz, 1H), 4.23 (d, J=7.5 Hz, 1H), 2.84 (qd, J=7.6, 1.1 Hz, 2H), 1.95-1.88 (m, 1H), 1.40 (td, J=7.5, 1.1 Hz, 3H), 1.03 (d, J=8.3 Hz, 2H), 0.67 (s, 2H). ¹²C NMR (126 MHz, CDCl3) δ 171.85 (s), 170.94 (s), 166.63 (s), 160.26 (d, J=251.6 Hz), 144.35 (s), 136.22 (s), 135.60 (s), 132.10 (d, ²J_{C-F}=8.4 Hz), 131.64 (s), 131.31 (d, ⁴J_{C-F}=2.3 Hz), 129.04 (s), 128.78 (s), 128.15 (s), 128.06 (s), 128.02 (s), 124.47 (s), 124.36 (d, ³J_{C-F}=3.5 Hz), 122.32 (s), 116.10 (d, 2J_{C-F}=21.6 Hz), 44.82 (s), 19.77 (s), 15.13 (s), 11.57 (s), 9.91 (s). HRMS (ESI/IT-TOF) m/z: [M+H]+Calcd for C₂₄ H₂₀ N₅O F 414.1725; found 414.1691.

### (R)-5-(8-chloro-6-(2-fluorophenyl)-4-methyl-4H-benzo[f]imidazo[1,5-a][1,4]diazepin-3-yl)-3-isopropyl-1,2,4-oxadiazole (MYM-IV-46)

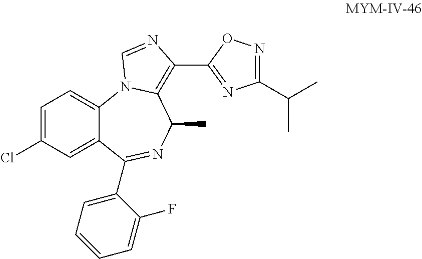 [SMILES: CC(C)c1noc(-c2ncn3c2[C@@H](C)N=C(c2ccccc2F)c2cc(Cl)ccc2-3)n1]

The ethyl ester, ethyl (R)-8-chloro-6-(2-fluorophenyl)-4-methyl-4H-benzo[f]imidazo[1,5-a][1,4]diazepine-3-carboxylate (1 g, 2.5 mmol) was dissolved in dry THF (20 mL) under argon and in a separate flask N-hydroxyisobutyrimidamide (1.05 g, 10 mmol) was treated with sodium hydride (60% dispersion in mineral oil, 0.065 g, 2.80 mmol) for an hour with molecular sieves, 3A. At that point the ethyl ester solution was added dropwise to the other flask and the reaction mixture, which resulted, was stirred at room temperature for 2 hours until the starting material was consumed (TLC, silica gel, EtOAc:Hex=50:50). The reaction mixture was quenched with aqueous NaHCO₃ solution (10 mL), diluted with water (50 mL) and extracted with EtOAc (3×100 mL). The combined organic layers were washed (aqueous 10% NaCl solution, 2×30 mL), dried (Na₂SO₄) and solvent was removed under reduced pressure. The solid residue was purified by flash column chromatography (silica gel, EtOAc:Hex=50:50) to afford the pure, white oxadiazole, MYM-IV-46 (940 mg, 86%). ¹H NMR (500 MHz, CDCl3) δ 8.05 (s, 1H), 7.72-7.63 (m, 1H), 7.60 (s, 2H), 7.46 (dd, J=12.5, 6.5 Hz, 1H), 7.27 (dd, J=17.0, 7.4 Hz, 2H), 7.06 (t, J=9.0 Hz, 1H), 6.73 (q, J=7.0 Hz, 1H), 3.18 (dt, J=13.4, 6.6 Hz, 1H), 1.40 (d, J=6.9 Hz, 6H), 1.36 (d, J=7.2 Hz, 3H). ¹³C NMR (126 MHz, CDCl3) δ 175.34 (s), 170.65 (s), 162.02 (d, ¹J_{C-F}=235.7 Hz), 139.10 (s), 136.19 (s), 135.51 (s), 133.50 (s), 132.95 (s), 132.21 (d, ⁴J_{C-F}=8.1 Hz), 132.08 (s), 131.35 (s), 131.12 (s), 130.90 (s), 130.33 (s), 129.80 (s), 128.38 (d, ³J_{C-F}=11.9 Hz), 125.14 (s), 124.62 (s), 124.29 (s), 123.44 (s), 116.29 (d, J_{C-F}=21.4 Hz), 50.33 (s), 26.76 (s), 20.62 (s), 20.56 (s), 14.99 (s). HRMS (ESI/IT-TOF) m/z: [M+H]+Calcd for C₂₃H₁₉N₅OFCl 436.13349; found 436.13746. % EE=>98% (HPLC). % Purity=>99% (HPLC).

### (R)-5-(8-chloro-6-(2-fluorophenyl)-4-methyl-4H-benzo[f]imidazo[1,5-a][1,4]diazepin-3-yl)-3-cyclopropyl-1,2,4-oxadiazole (MYM-IV-47)

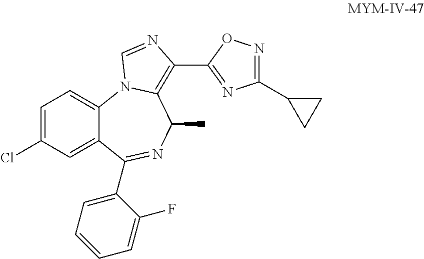 [SMILES: C[C@H]1N=C(c2ccccc2F)c2cc(Cl)ccc2-n2cnc(-c3nc(C4CC4)no3)c21]

N=Hydroxycyclopropanecarboximidamide (1.07 g, 10 mmol) was treated with sodium hydride (60% dispersion in mineral oil, 0.066 g, 2.75 mmol) for an hour with molecular sieves, 3A under argon atmosphere and then a solution of ethyl (R)-8-chloro-6-(2-fluorophenyl)-4-methyl-4H-benzo[f]imidazo[1,5-a][1,4]diazepine-3-carboxylate (1 g, 2.5 mmol, 20 mL THF) was added to the reaction mixture dropwise and stirred for 2 hours until consumption of starting material. The mixture was then quenched (20 mL saturated aqueous NaHCO₃), diluted (water, 50 mL) and extracted (EtOAc, 3×100 mL). The combined organic layer was washed (10% aqueous. NaCl solution 2×50 mL), dried (Na₂SO₄), the solvents were evaporated and the residue was purified by silica gel flash chromatography (EtOAc:Hex=50:50) to get pure MYM-IV-47 (white powder, 0.99 g, 91%). ¹H NMR (500 MHz, CDCl3) δ 8.04 (s, 1H), 7.65 (d, J=9.1 Hz, 1H), 7.60 (s, 2H), 7.46 (td, J=7.4, 1.5 Hz, 1H), 7.26 (dd, J=14.1, 6.4 Hz, 2H), 7.05 (t, J=9.2 Hz, 1H), 6.66 (q, J=7.1 Hz, 1H), 4.39 (d, J=6.2 Hz, 1H), 2.20-2.11 (m, 2H), 1.33 (d, J=7.2 Hz, 3H), 1.19-1.10 (m, 2H), 1.06 (dd, J=8.3, 2.2 Hz, 2H). ¹³C NMR (126 MHz, CDCl3) δ 172.64 (s), 170.48 (s), 162.94 (s), 160.07 (d, ¹J_{C-F}=249.6 Hz), 139.11 (s), 136.16 (s), 135.48 (s), 133.51 (s), 132.92 (s), 132.55 (d, ³J_{C-F}=8.0 Hz), 132.20 (d, ⁴J_{C-F}=7.6 Hz), 132.05 (s), 131.10 (s), 130.88 (s), 130.32 (s), 129.80 (s), 128.36 (d, ²J_{C-F}=12.0 Hz), 125.01 (s), 124.61 (s), 124.28 (s), 123.41 (s), 116.28 (d, ²J_{C-F}=21.4 Hz), 50.29 (s), 14.98 (s), 7.76 (s), 6.90 (s). HRMS (ESI/IT-TOF) m/z: [M+H]+Calcd for C₂₃ H₁₇ N₅OF Cl 434.11784; found 434.12100. % EE=>98% (HPLC). % Purity=>99% (HPLC).

### (R)-5-(8-Cyclopropyl-6-(2-fluorophenyl)-4-methyl-4H-benzo[f]imidazo[1,5-a][1,4]diazepin-3-yl)-3-ethyl-1,2,4-oxadiazole (GL-IV-03)

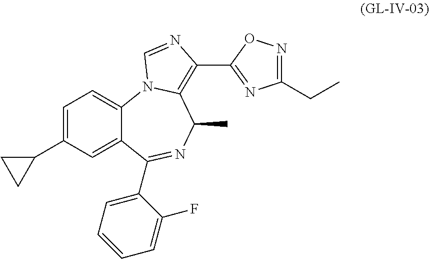 [SMILES: CCc1noc(-c2ncn3c2[C@@H](C)N=C(c2ccccc2F)c2cc(C4CC4)ccc2-3)n1]

To a solution of (R)-5-(8-bromo-6-(2-fluorophenyl)-4-methyl-4H-benzo[f]imidazo[1,5-a][1,4]diazepin-3-yl)-3-ethyl-1,2,4-oxadiazole (1.1 g, 2.38 mmol), tri(O-tolyl)phosphine (85.5 mg, 0.28 mmol), cyclopropyl boronic acid (0.724 g, 8.43 mmol) and potassium phosphate (2.56 g, 12.1 mmol) in toluene (30 mL) and water (0.65 mL), Pd(OAc)₂ (31.5 mg, 0.14 mmol) was added under Ar at room temperature to form an orange cloudy solution. A reflux condenser was attached. The mixture was allowed to stir at room temperature for 5 minutes until the color of the solution turned yellow, as an indication of the formation of the Pd complex generated in situ. The mixture was then placed into a pre-heated oil bath at 100° C. After 2 hours, the reaction progress was complete on analysis by TLC (silica gel) and it was then cooled to room temperature. Then water (20 mL) was added and the mixture was extracted with EtOAc (3×25 mL), after which the filtrate was washed with brine (20 mL), dried (Na₂SO₄) and concentrated under reduced pressure. The black residue which resulted was purified by a wash column (silica gel, dichloromethane and 5% MeOH) to afford the title compound as a white solid (815.5 mg, 81.6%): ¹H NMR (300 MHz, CDCl3) δ 7.98 (s, 1H), 7.46 (d, J=8.3 Hz, 2H), 7.38-7.25 (m, 1H), 7.13 (t, J=7.5 Hz, 2H), 6.93 (t, J=9.1 Hz, 2H), 6.63 (q, J=7.0 Hz, 1H), 2.73 (q, J=7.6 Hz, 2H), 1.84-1.65 (m, 1H), 1.27 (dd, J=14.1, 6.6 Hz, 6H), 0.90 (d, J=8.3 Hz, 2H), 0.54 (dd, J=10.1, 4.8 Hz, 2H); ¹³C NMR (75 MHz, CDCl3) δ 171.70 (s), 170.95 (s), 164.32 (s), 160.06 (d, J=250.5 Hz), 144.12 (s), 139.17 (s), 136.28 (s), 131.72 (s), 131.62 (s), 131.17 (s), 129.05 (t, J=6.2 Hz), 128.61 (s), 127.96 (s), 127.20 (s), 124.36 (d, J=8.0 Hz), 122.68 (s), 121.94 (s), 115.94 (d, J=21.6 Hz), 50.13 (s), 19.69 (s), 15.02 (s), 14.68 (s), 11.49 (s), 9.89 (s); HRMS (ESI/IT-TOF) m/z: [M+H] Calcd for C₂₅H₂₃FNO 428.1881, found 428.1889.

### (R)-5-(8-Bromo-6˜(2-fluorophenyl)-4-methyl-4H-benzo[f]imidazo[1,5-a][1,4] diazepin-3-yl)-3-methyl-1,2,4-oxadiazole (GL-III-60)

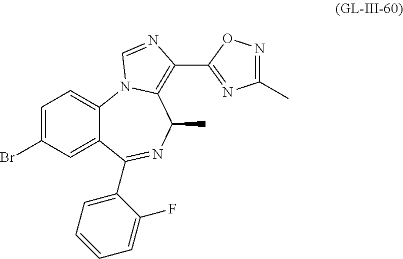 [SMILES: Cc1noc(-c2ncn3c2[C@@H](C)N=C(c2ccccc2F)c2cc(Br)ccc2-3)n1]

The title compound was prepared from ethyl (R)-8-bromo-6-(2-fluorophenyl)-4-methyl-4H-benzo[f]imidazo[1,5-a][1,4]diazepine-3-carboxylate (6 g, 13.5 mmol) following the general procedure for oxadiazoles with N′-hydroxyacetimidamide (3 g, 40.5 mmol) and NaH (60% dispersion in mineral oil, 0.8 g, 14.9 mmol). The crude residue was purified by flash column chromatography (silica gel, EtOAc/Hexane 3:2) to yield the title compound as a white powder (5.6 g, 91.8%): ¹H NMR (500 MHz, CDCl₃) δ 8.08 (s, 1H), 7.76 (dd, J=8.5, 1.9 Hz, 1H), 7.61 (t, J=7.0 Hz, 1H), 7.55 (d, J=8.6 Hz, 1H), 7.52-7.42 (m, 2H), 7.26 (td, J=7.6, 0.9 Hz, 1H), 7.11-6.99 (m, 1H), 6.74 (q, J=7.3 Hz, 1H), 2.45 (s, 3H), 1.35 (d, J=7.3 Hz, 3H); ¹³C NMR (126 MHz, CDCl₃) δ 170.69 (s), 167.46 (s), 161.09 (s), 161.06 (d, J=495.1 Hz), 139.12 (s), 136.31 (s), 135.16 (s), 133.37 (s), 133.25 (s), 132.36 (d, J=7.7 Hz), 131.26 (s), 131.02 (s), 128.15 (d, J=11.0 Hz), 124.87 (s), 124.61 (d, J=3.3 Hz), 123.71 (s), 121.26 (s), 116.29 (d, J=21.4 Hz), 50.15 (s), 14.98 (s), 11.69 (s); HRMS (ESI/IT-TOF) m/z: [M+H] Calcd for C₂₁H₁₆BrFN₅O 452.0517, found 452.0542.

### (R)-5-(6-(2-chlorophenyl)-8-ethynyl-4-methyl-4H-benzo[f]imidazo[1,5-a][1,4]diazepin-3-yl)-3-ethyl-1,2,4-oxadiazole (PM-II-84E)

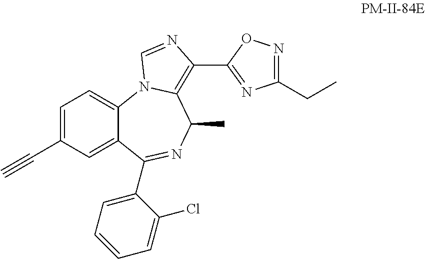 [SMILES: C#Cc1ccc2c(c1)C(c1ccccc1Cl)=N[C@H](C)c1c(-c3nc(CC)no3)ncn1-2]

The title compound was prepared from (R)-ethyl 6-(2-chlorophenyl)-8-ethynyl-4-methyl-4H-benzo[f]imidazo[1,5-a][1,4]diazepine-3-carboxylate. N′-hydroxypropionimidamide (0.58 g, 6.5 mmol) was treated with NaH (0.043 g, 1.70 mmol) for 1 hour at room temperature in THF. Then the ethyl ester (0.658 g, 1.6 mmol) dissolved in THF was added dropwise for 30 minutes. After 4 hours the reaction was complete. The crude residue was purified by flash column chromatography (silica gel, EtOAc/Hexane 3:2) to yield pure title compound as a white powder (0.58 g, 83%). ¹H NMR (500 MHz, CDCl3): δ 8.06 (s, 1H), 7.74 (dd, J=16.6, 8.1 Hz, 1H), 7.62 (d, J=8.3 Hz, 1H), 7.55-7.44 (m, 1H), 7.40 (dd, J=5.6, 3.5 Hz, 3H), 7.29 (s, 1H), 6.71 (Q, J=12.5, 7.1 Hz, 1H), 3.17 (s, 1H), 2.83 (q, J=7.3 Hz, 2H), 1.40 (d, J=3.4 Hz, 3H), 1.38 (t, J=5.9 Hz, 3H). ¹³C NMR (126 MHz, CDCl3): δ 171.90 (s), 139.51 (s), 139.18 (s), 136.18 (s), 135.32 (s), 134.73 (s), 133.91 (s), 132.38 (s), 130.81 (S), 130.67 (s), 130.16 (s), 129.47 (s), 129.20 (s), 128.34 (s), 127.14 (s), 125.15 (s), 122.02 (s), 121.86 (s), 81.37 (s), 79.90 (s), 50.28 (s), 19.76 (s), 15.11 (s), 11.53 (s) %). HRMS (ESI/IT-TOF) m/z: [M+H]⁺ Calcd for C₂₄H₁₈ClN₅O 428.127; found 428.129.

### (R)-3-cyclopropyl-5-(8-ethynyl-6-(2-fluorophenyl)-4-methyl-4H-benzo[f]imidazo [1,5-a][1,4]diazepin-3-yl)-1,2,4-oxadiazole (MYM-V-28)

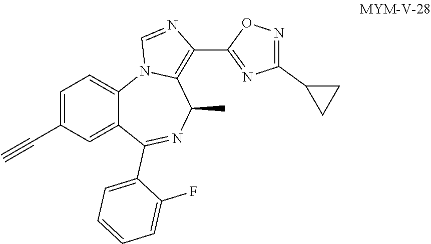 [SMILES: C#Cc1ccc2c(c1)C(c1ccccc1F)=N[C@H](C)c1c(-c3nc(C4CC4)no3)ncn1-2]

After treating 1 h with sodium hydride (60% dispersion in mineral oil, 0.068 g, 2.75 mmol), the solution of N′-hydroxycyclopropanecarboximidamide (1.03 g, 10.3 mmol) was added to the stirred solution of solution of ethyl (R)-8-ethynyl-6-(2-fluorophenyl)-4-methyl-4H-benzo[f]imidazo[1,5-a][1,4]diazepine-3-carboxylate (1 g, 2.58 mmol) in dry THF (20 mL). The mixture was then stirred for 2 hr until consumption of starting material. The mixture was the quenched with saturated aq NaHCO₃ (20 mL), diluted with water (10 mL), and extracted with EtOAc (3×20 mL). The combined organic layers were washed (10% aq. NaCl solution 2×50 mL) and dried (Na₂SO₄). The solvents were evaporated and the residue was purified by silica gel flash chromatography to get pure MYM-V-28 as a white powder (896 mg, 82%). R_{f}=0.6 for MYM-V-28 and R_{f}=0.4 for SH-2′F—R—CH₃ (silica gel, 70% EtOAc-hexane). ¹H NMR (500 MHz, CDCs) δ 8.07 (s, 1H), 7.75 (dd, J=14.6, 7.3 Hz, 1H), 7.63 (t, J=13.9 Hz, 2H), 7.51-7.40 (m, 2H), 7.26 (d, J=7.5 Hz, 1H), 7.06 (t, J=9.3 Hz, 1H), 6.68 (q, J=7.1 Hz, 1H), 3.18 (s, 1H), 2.16 (ddd, J=16.5, 8.2, 3.9 Hz, 1H), 1.34 (d, J=7.3 Hz, 2H), 1.16 (dd, J=11.9, 9.7 Hz, 2H), 1.07 (dd, J=8.4, 2.3 Hz, 2H). ¹³C NMR (126 MHz, CDCl₃) δ 172.67 (s), 170.43 (S), 163.76 (s), 160.10 (d, J=250.9 Hz), 139.08 (s), 136.24 (s), 135.52 (s), 134.24 (s), 133.70 (d, J=20.2 Hz), 132.30 (d, J=8.7 Hz), 131.19 (s), 129.38 (d, J=2.6 Hz), 128.24 (d, J=5.1 Hz), 125.05 (s), 124.58 (d, J=3.3 Hz), 123.01 (s), 122.24 (s), 121.95 (s), 116.29 (d, J=21.4 Hz), 81.29 (s), 79.97 (s), 50.15 (s), 15.02 (s), 7.76 (s), 6.90 (s). HRMS (ESI/IT-TOF) m/z: [M+H]+Calcd for C₂₅H₁₈N₅OF 424.15681.

### (R)-5-(6-(2-chlorophenyl)-8-ethynyl-4-methyl-4H-benzo[f]imidazo[1,5-a][1,4]diazepin-3-yl)-3-methyl-1,2,4-oxadiazole (PM-III-57R)

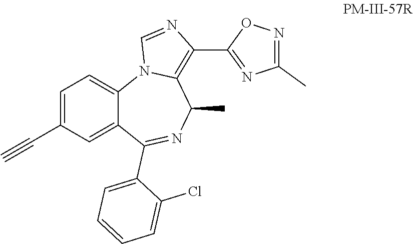 [SMILES: C#Cc1ccc2c(c1)C(c1ccccc1Cl)=N[C@H](C)c1c(-c3nc(C)no3)ncn1-2]

Ethyl (R)-6-(2-chlorophenyl)-8-ethynyl-4-methyl-4H-benzo[f]imidazo[1,5-a][1,4]diazepine-3-carboxylate (1 g, 2.4 mmol) was dissolved in anhydrous THF (25 mL) at room temperature. In a separate flask N′-hydroxymethylcarboximidamide (9.6 mmol) and NaH (60% dispersion in mineral oil, 2.6 mmol) were mixed at room temperature in THF (20 mL) and stirred for 1 hour. The solution of ethyl (R)-6-(2-chlorophenyl)-8-ethynyl-4-methyl-4H-benzo[f]imidazo[1,5-a][1,4]diazepine-3-carboxylate was then added to the suspension of N′-hydroxymethylcarboximidamide and NaH dropwise at room temperature and stirred for 4 hours. The progress of the reaction was monitored by TLC. To quench the reaction, saturated aqueous NaHCO₃ (25 mL) was added, followed by dilution with water (12 mL), and extraction with EtOAc (2×20 mL). The combined organic layers were washed (10% aq. NaCl solution 3×50 mL) and dried (Na₂SO₄). The solvents were evaporated under reduced pressure, and the residue was purified by silica gel flash chromatography to get pure PM-III-57R as a white powder (850 mg, 83%). ¹H NMR (500 MHz, CDCl₃) δ 8.06 (s, 1H), 7.73 (d, J=8.0 Hz, 1H), 7.62 (d, J=8.3 Hz, 1H), 7.56-7.48 (m, 1H), 7.40 (dd, J=5.8, 3.4 Hz, 2H), 7.33 (d, J=37.0 Hz, 2H), 6.73 (q, 1H), 3.17 (s, 1H), 2.47 (s, 3H), 1.39 (d, J=6.6 Hz, 3H). ¹³C NMR (75 MHz, CDCl₃) δ 170.74 (s), 167.42 (s), 165.82 (s), 139.43 (s), 139.21 (s), 136.23 (s), 135.32 (s), 134.66 (s), 133.84 (s), 132.36 (s), 130.80 (s), 130.70 (s), 130.12 (s), 129.46 (s), 129.12 (s), 127.11 (s), 122.05 (s), 121.90 (s), 81.35 (s), 79.95 (s), 50.21 (s), 15.10 (s), 11.66 (s).

### (S)-5-(6-(2-chlorophenyl)-8-ethynyl-4-methyl-4H-benzo[f]imidazo[1,5-a][1,4]diazepin-3-yl)-3-methyl-1,2,4-oxadiazole

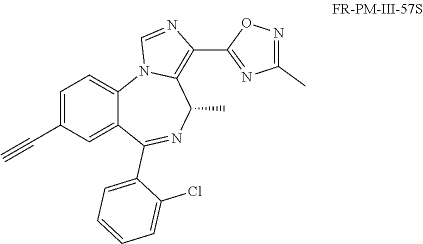 [SMILES: C#Cc1ccc2c(c1)C(c1ccccc1Cl)=N[C@@H](C)c1c(-c3nc(C)no3)ncn1-2]

Ethyl (S)-6-(2-chlorophenyl)-8-ethynyl-4-methyl-4H-benzo[f]imidazo[1,5-a][1,4]diazepine-3-carboxylate (1 g, 2.4 mmol) was dissolved in anhydrous THF (25 mL) at room temperature. In a separate flask N-hydroxymethylcarboximidamide (9.6 mmol) and NaH (60% dispersion in mineral oil, 2.6 mmol) were mixed at room temperature in THF (20 mL) and stirred for 1 hour. The solution of ethyl (S)-6-(2-chlorophenyl)-8-ethynyl-4-methyl-4H-benzo[f]imidazo[1,5-a][1,4]diazepine-3-carboxylate was then added to the suspension of N′-hydroxynethylcarboximidamide and NaH dropwise at room temperature and stirred for 4 hours. The progress of the reaction was monitored by TLC. To quench the reaction saturated aqueous NaHCO₃ (25 mL) was added, followed by dilution with water (12 mL), and extraction with EtOAc (2×20 mL). The combined organic layers were washed (10% aq. NaCl solution 3×50 mL) and dried (Na₂SO₄). The solvents were evaporated under reduced pressure, and the residue was purified by silica gel flash chromatography to get pure PM-FR-III-57S as a white powder (890 mg, 87%)¹H NMR (500 MHz, CDCl₃) δ 8.06 (s, 1H), 7.72 (d, J=8.0 Hz, 1H), 7.62 (d, J=8.3 Hz, 1H), 7.59-7.48 (m, 1H), 7.39 (dd, J=8.3, 4.5 Hz, 2H), 7.37-7.29 (m, 2H), 6.72 (q, 1H), 3.17 (s, 1H), 2.46 (s, 3H), 1.39 (d, J=6.9 Hz, 3H). ¹³C NMR (126 MHz, CDCl₃) δ 170.74 (s), 167.44 (s), 165.84 (s), 139.46 (s), 139.22 (s), 136.23 (s), 135.31 (s), 134.70 (s), 133.87 (s), 132.39 (s), 130.80 (s), 130.70 (s), 130.14 (s), 129.50 (s), 127.12 (s), 124.98 (s), 122.02 (s), 121.88 (s), 81.37 (s), 79.92 (s), 50.22 (s), 15.08 (s), 11.66 (s).

### Y-Maze Alternation Task

Young Animals and chronic stress procedure: C57/Bl6 mice, males and females at 8 weeks old and are kept in normal housing conditions (12 h light ON cycle starting at 7 am/water and food ad libitum) for one week. During this week, animals are single housed and handled to reduce the anxiety-like response toward the experimenter. To induce a working memory deficit, mice are subjected to a chronic restraint stress protocol (CRS). They are placed in a 50 mL Falcon® tube, twice a day, for 1 hour during their diurnal cycle. The time of occurrence of the stress is randomized every day to avoid predictability. CRS is not applied on testing days. Y-maze test occurs 15 hours minimum after the last stressor.

Young versus Old Animals testing: C57/Bl6 male mice at 10 months-old and are kept in normal housing conditions (12 h light ON cycle starting at 7 am/water and food ad libitum) until they reach the age of 22 months. A younger cohort of 8 week old animals is used as controls for the experiment. Before testing animals are handled to reduce the anxiety-like response toward the experimenter. Normal aging induced-working memory deficits are assessed using the Y-maze test as described below.

Protocol: Mice are first habituated to the Y-maze apparatus and distal cues during 2 consecutive days over a 10 min free exploration session (one session per day). The next day, animals perform a training session consisting in seven successive trials separated by a 30 s inter-trial interval (ITI); during this training session, mice are familiarized with the experimental procedure (opening and closing of the doors and confinement into the goal arms). For each trial, mice remain in the start-box for a 30 s ITI. Then, the door is opened and the mouse is allowed to enter freely one of the two goal arms; the chosen arm is closed and the choice is recorded. After a 30 s confinement period into the chosen arm, the mouse is removed and brought back to the start-box for a second trial identical to the first one and this procedure is repeated over the series of trials.

The same general procedure used in the training session is implemented 24 h later, except that the ITI was lengthened to 90 s. For this experiment, animals are acutely injected i.p. with vehicle or drug, 30 minutes before the beginning of the test. To dissociate memory deficits from an eventual progressive loss of motivation to alternate over the series, an 8th trial is added to the series which is separated from the 7th trial by a shorter ITI (5 s). All animals failing to alternate at the 8th trial are excluded. The mean alternation rate is calculated and is expressed in percentage (number of alternation done by the animal/total number of alternation possible×100)±SEM. The percentage of alternation during the entire task is considered as an index of working memory performance (50% of being a random alternation rate). Factorial ANOVA is applied to the data to reveal differences between groups. If the ANOVA is significant for group effect (p<0.05), Scheffe post-hoc analysis are conducted to identify which groups are different from each other.

Drug preparation and administration: All drugs are diluted in a vehicle solution containing 85% H₂O, 14% propylene glycol (Sigma Aldrich) and 1% Tween 80 (Sigma Aldrich). Working solutions are prepared at a concentration of 10 mg/mL and administered i.p. adjusted to the body weight of each animal. Doses used are either 1, 5, 10 or 20 mg/kg. For all Y maze experiments, animals are acutely injected i.p. with vehicle or drug, 30 minutes before the beginning of the test.

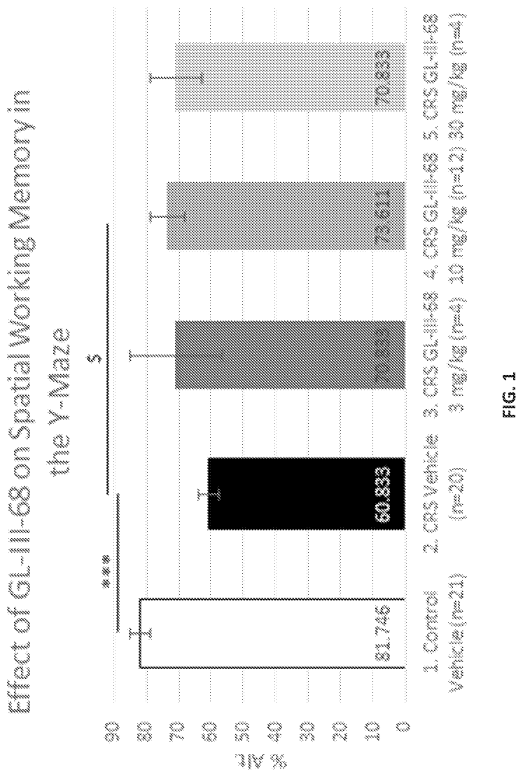

Results of the Y-Maze alternation task after treatment with GL-III-68: Mice subjected to CRS were injected with GL-III-68 (3 mg/kg, 10 mg/kg, or 30 mg/kg i.p., 30 min before testing) and tested for spatial working memory in the Y-maze. ANOVA found an overall effect of intervention (p<0.001). Stress significantly reduced alternation rate (p<0.001) and this impairment was reversed by treatment with 10 mg/kg GL-III-60 ($ p<0.05) (FIG. 1). These results suggest a potential indication of this compound to reverse cognitive deficits.

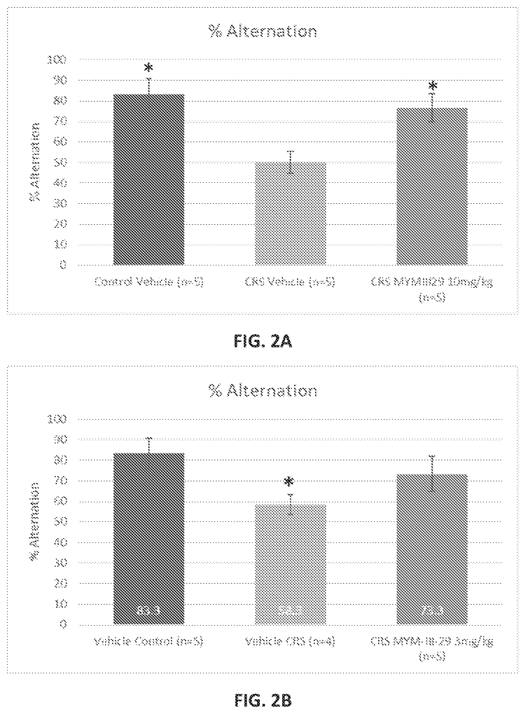

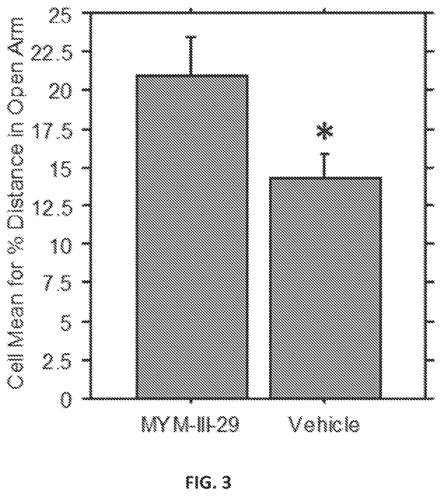

Results of the Y-Maze alternation task after treatment with MYM-III-29: Data analysis (FIGS. 2A and 2B) showed a significant difference between groups (ANOVA: F(2,12)=7.3, p<0.01). Fisher's PLSD post hoc analysis shows that chronic restraint stress (CRS) induces significant reduction of alternation rate (p<0.01) compared to Control Vehicle mice. This effect is reversed (p<0.05) by acute administration of MYM-III-29, 10 mg/kg, 30 minutes prior to testing. MYM-III-29 has a significant pro-cognitive effect at 10 mg/kg (p<0.05, compared to the Control-Vehicle group). These results suggest a potential indication of this compound to reverse cognitive deficits.

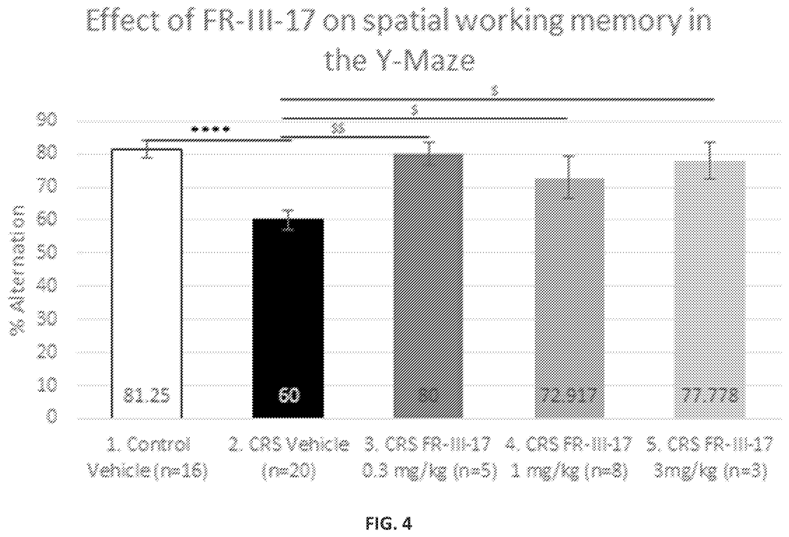

Results of the Y-Maze alternation task after treatment with FR-III-17: Mice subjected to chronic restraint stress were injected with FR-III-17 (0.3 mg/kg, 1 mg/kg and 3 mg/kg i.p., 30 min before testing) and tested for spatial working memory in the Y-maze. Analysis of variance found an overall effect (p<0.001) and post hoc analysis revealed that stress significantly reduced alternation rate (*** p<0.0001), this deficit was significantly reversed by 0.3 mg/kg ($$ p<0.01), 1 mg/kg ($ p<0.05) or 3 mg/kg (p<0.05) of FR-III-17 (FIG. 4). These results suggest a potential indication of this compound to reverse cognitive deficits.

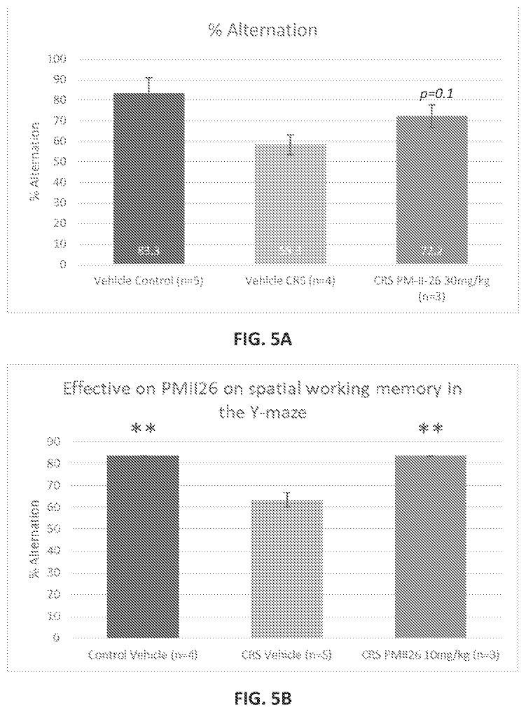

Results of the Y-Maze alternation task after treatment with PM-1-26: Data analysis (FIGS. 5A and 5B) showed impaired alternation by CRS, which is reversed by PM-II-26 treatment at 10 mg/kg (p<0.001) and a trend was observed at 30 mg/kg (p=0.1). These results suggest a potential indication of this compound to reverse cognitive deficits.

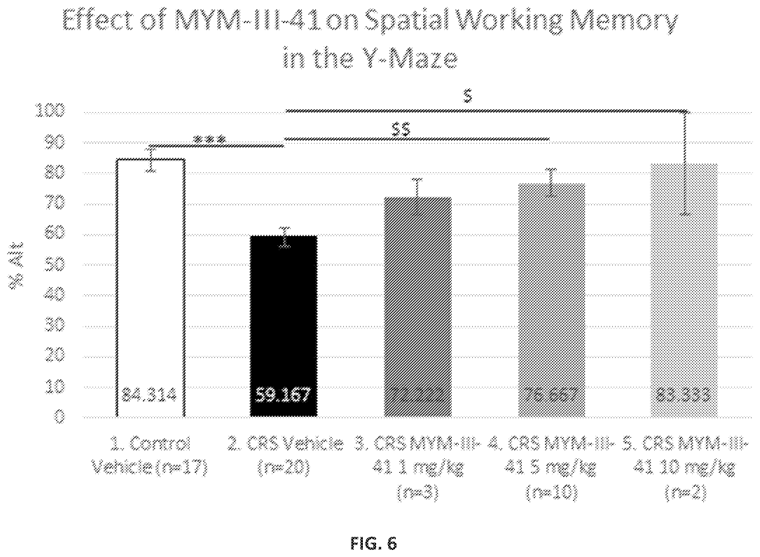

Results of the Y-Maze alternation task after treatment with MYM-III-41: Mice subjected to CRS were injected with MYM-III-41 (1 mg/kg, 5 mg/kg, or 10 mg/kg i.p., 30 min before testing) and tested for spatial working memory in the y-maze. ANOVA found an overall effect of intervention (p<0.0001). Stress significantly reduced alternation rate (p<0.001) and this impairment was reversed by treatment with 5 mg/kg MYM-III-41 (p<0.01) and 10 mg/kg MYM-III-41 (p<0.05) (FIG. 6). These results suggest a potential indication of this compound to reverse cognitive deficits.

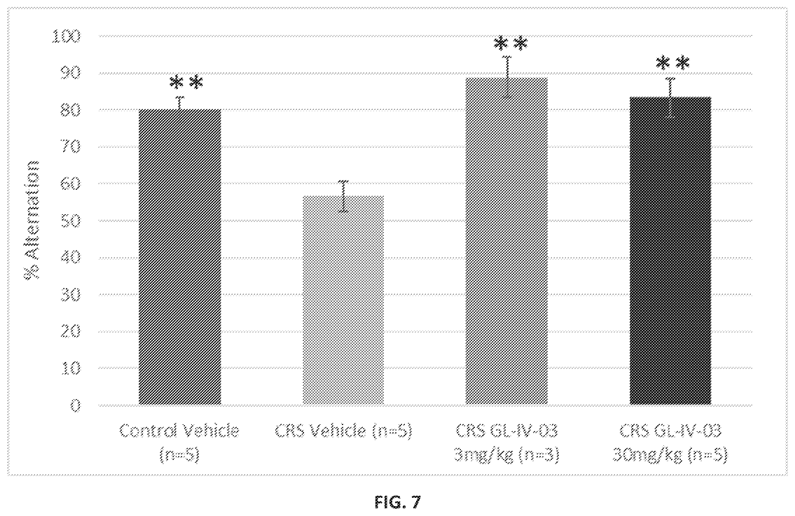

Results of the Y-Maze alternation task after treatment with GL-IV-03: Data analysis (FIG. 7) showed a significant difference between groups (ANOVA: F(3,14)=9.7, p<0.001). Fisher's PLSD post hoc analysis shows that chronic restraint stress (CRS) induces significant reduction of alternation rate (p<0.01) compared to Control Vehicle mice. This effect is reversed by acute administration of GL-IV-03, 3 mg/kg (p<0.01) and 30 mg/kg (p<0.01), 30 minutes prior to testing. These results suggest a potential indication of this compound to reverse cognitive deficits.

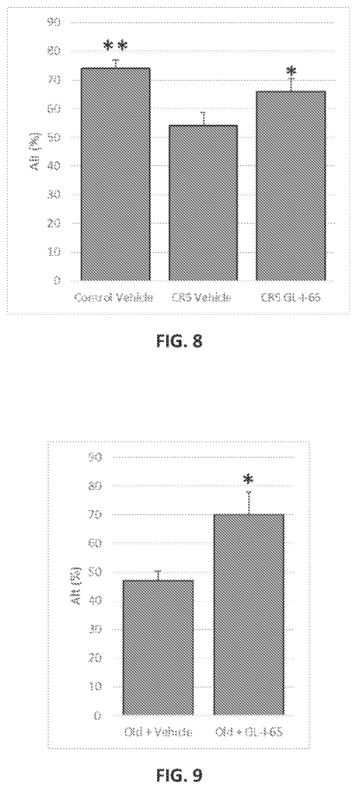

Results of the Y-Maze alternation task after treatment with GL-1-65: Data analysis (FIGS. 8 and 9) showed a significant difference between groups (ANOVA: F(2,28)=5.3, p<0.05). Fisher's PLSD post hoc analysis shows that chronic restraint stress (CRS) induces significant reduction of alternation rate (p<0.01) compared to Control Vehicle mice. This effect is reversed (p<0.05) by acute administration of GL-1-65, 10 mg/kg, 30 minutes prior to testing (FIG. 8). Similar improvement was observed in old mice, where acute administration of GL-1-65 (10 mg/kg), 30 minutes prior to testing improved alternation rate in old mice (p<0.05) (FIG. 9). These results suggest a potential indication of this compound to reverse cognitive deficits.

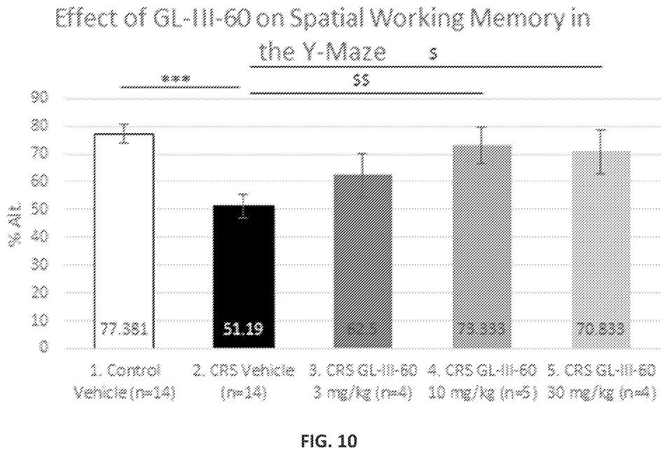

Results of the Y-Maze alternation task after treatment with GL-III-60: Mice subjected to CRS were injected with GL-III-60 (3 mg/kg, 10 mg/kg, or 30 mg/kg i.p., 30 min before testing) and tested for spatial working memory in the Y-maze. ANOVA found an overall effect of intervention (p<0.001). Stress significantly reduced alternation rate (p<0.001) and this impairment was reversed by treatment with 10 mg/kg GL-III-60 ($$ p<0.01) and 30 mg/kg ($ p<0.05) (FIG. 10). These results suggest a potential indication of this compound to reverse cognitive deficits.

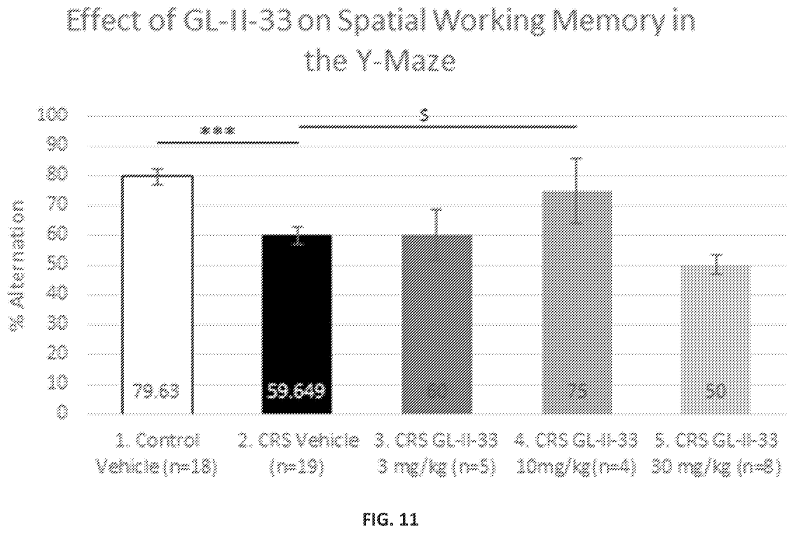

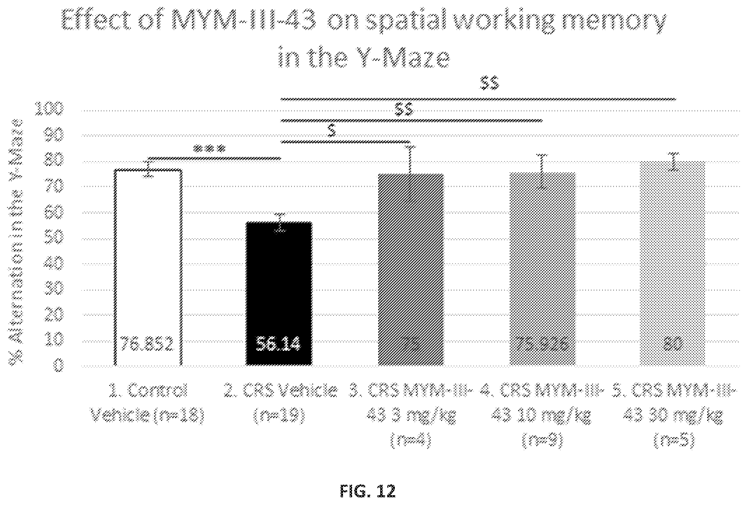

Results of the Y-Maze alternation task after treatment with GL-II-33: Mice subjected to CRS were injected with GL-II-33 (3 mg/kg, 10 mg/kg, or 30 mg/kg i.p., 30 min before testing) and tested for spatial working memory in the Y-maze. ANOVA found an overall effect of intervention (p<0.001). Stress significantly reduced alternation rate (p<0.001) and this impairment was reversed by treatment with 10 mg/kg GL-II-33 ($ p<0.05) and but not 3 mg/kg or 30 mg/kg (FIG. 11). These results suggest a potential indication of this compound to reverse cognitive deficits.

Results of the Y-Maze alternation task after treatment with MYM-III-43: Mice subjected to CRS were injected with MYM-III-43 (3 mg/kg, 10 mg/kg and 30 mg/kg by i.p. injection, 30 min before testing) and tested for spatial working memory in the Y-maze. ANOVA found a significant overall effect of manipulation (p<0.001), and post hoc testing revealed a significant effect of stress compared to controls (*** p<0.001) and this was reversed by administration of MYM-III-43 at 3 mg/kg ($<0.05), 10 mg/kg $$ ($$ p<0.01) and 30 mg/kg ($$ p<0.01) (FIG. 12). These results suggest a potential indication of this compound to reverse cognitive deficits.

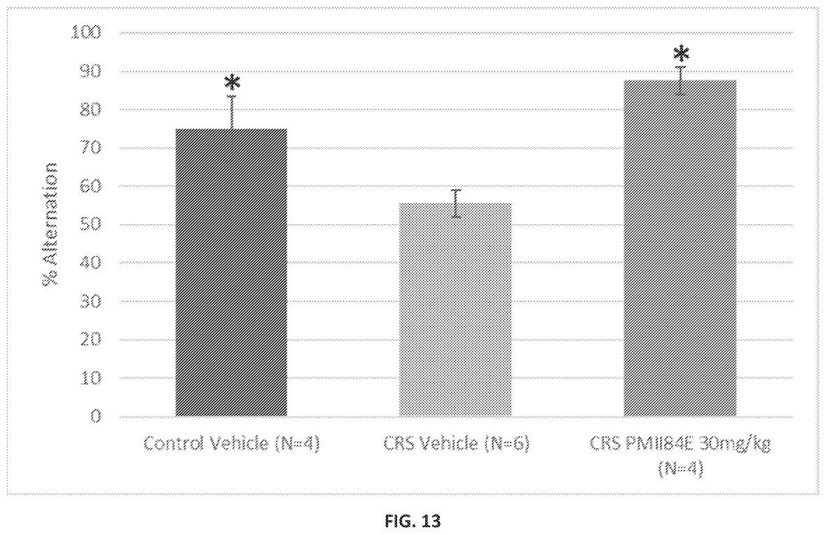

Results of the Y-Maze alternation task after treatment with PM-II-84E: Data analysis (FIG. 13) showed a significant difference between groups (ANOVA: F(2,11)=10, p<0.01). Fisher's PLSD post hoc analysis shows that chronic restraint stress (CRS) induces significant reduction of alternation rate (p<0.05) compared to Control Vehicle mice. This effect is reversed by acute administration of PM-II-84E, 30 mg/kg (p<0.05), 30 minutes prior to testing. These results suggest a potential indication of this compound to reverse cognitive deficits.

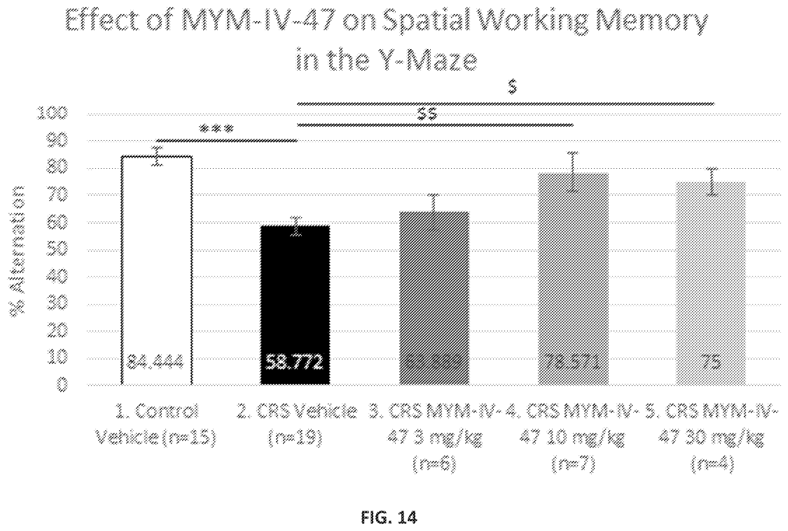

Results of the Y-Maze alternation task after treatment with MYM-IV-47: Mice subjected to CRS were injected with MYM-IV-47 (3 mg/kg, 10 mg/kg and 30 mg/kg by i.p. injection, 30 min before testing) and tested for spatial working memory in the Y-maze. ANOVA found a significant overall effect of manipulation (p<0.001), and post hoc testing revealed a significant effect of stress compared to controls (*** p<0.001) and this was significantly reversed by administration of MYM-IV-47 at 10 mg/kg ($$<0.01) and 30 mg/kg MYM-IV-47 rate ($ p<0.05), there was no significant effect at 3 mg/kg (FIG. 14). These results suggest a potential indication of this compound to reverse cognitive deficits.

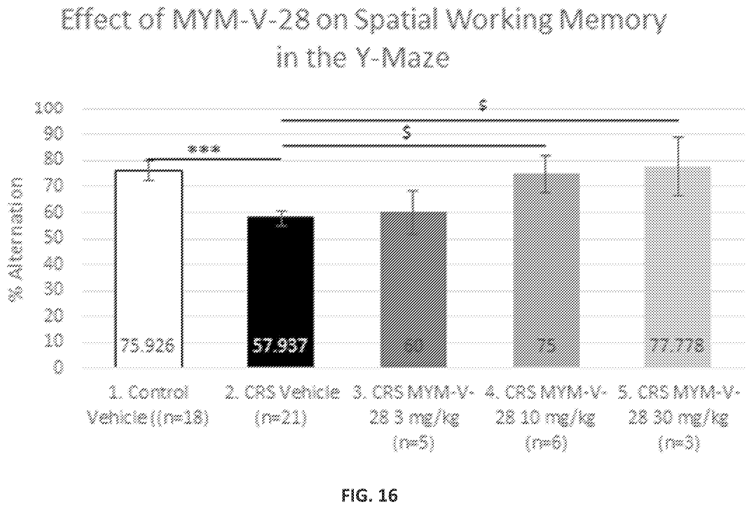

Results of the Y-Maze alternation task after treatment with MYM-V-28: Mice subjected to CRS were injected with MYM-V-28 (3 mg/kg, 10 mg/kg and 30 mg/kg by i.p. injection, 30 min before testing) and tested for spatial working memory in the Y-maze. ANOVA found a significant overall effect of manipulation (p<0.05), and post hoc testing revealed a significant effect of stress compared to controls (*** p<0.001) and this was significantly reversed by administration of MYM-V-28 at 10 mg/kg ($<0.05) and 30 mg/kg ($ p<0.05), but not at 3 mg/kg (FIG. 16). These results suggest a potential indication of this compound to reverse cognitive deficits.

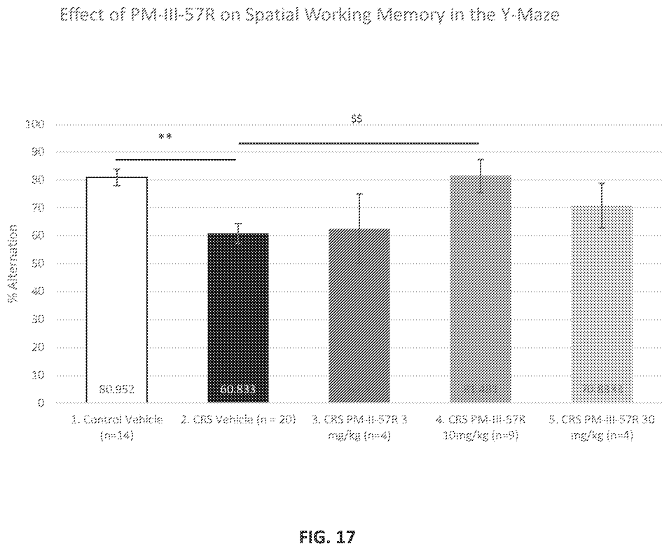

Results of the Y-Maze alternation task after treatment with PM-III-57R: Mice subjected to CRS were injected with PM-III-57R (3 mg/kg, 10 mg/kg and 30 mg/kg by i.p. injection, 30 min before testing) and tested for spatial working memory in the Y-maze. ANOVA found a significant overall effect of manipulation (p<0.01), and post hoc testing revealed a significant effect of stress compared to controls (** p<0.01) and this was significantly reversed by administration of PM-III-57R at 10 mg/kg ($$<0.01) but not at 3 mg/kg or 30 mg/kg (FIG. 17). These results suggest a potential indication of this compound to reverse cognitive deficits.

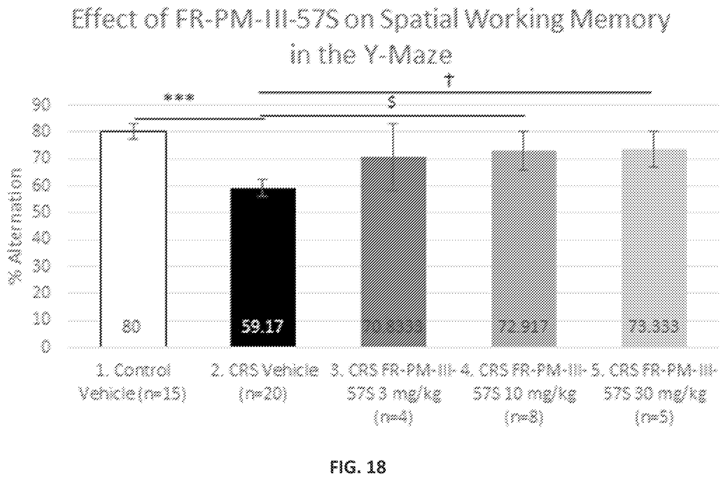

Results of the Y-Maze alternation task after treatment with FR-PM-III-57S: Mice subjected to CRS were injected with FR-PM-III-57S (3 mg/kg, 10 mg/kg, 30 mg/kg by i.p. injection, 30 min before testing) and tested for spatial working memory in the Y-maze. ANOVA found a significant effect of manipulation (p<0.01), and post hoc testing revealed a significant effect of stress compared to controls (* p<0.001). This was significantly reversed by administration of FR-PM-III-57S at 10 mg/kg ($<0.05), there was also a trend level effect of FR-PM-III-57S at 30 mg/kg (†<0.10) (FIG. 18). These results suggest a potential indication of this compound to reverse cognitive deficits.

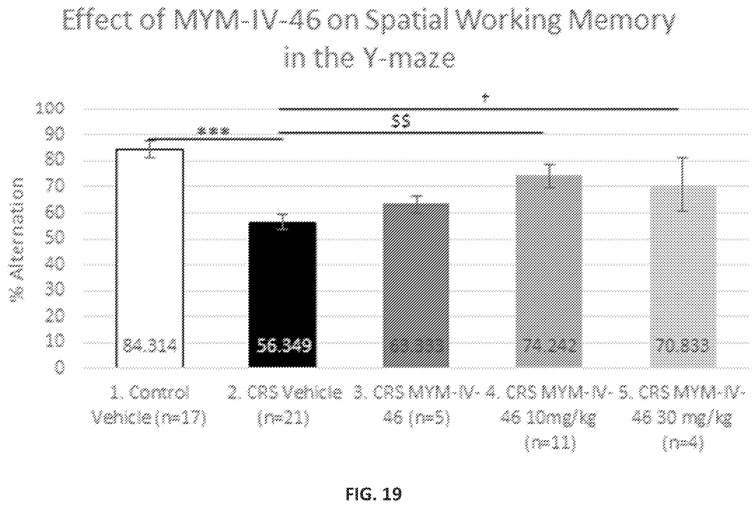

Results of the Y-Maze alternation task after treatment with MYM-IV-46: Mice subjected to CRS were injected with MYM-IV-46 (3 mg/kg, 10 mg/kg and 30 mg/kg by i.p. injection, 30 min before testing) and tested for spatial working memory in the y-maze. ANOVA found a significant overall effect of manipulation (p<0.001), and post hoc testing revealed a significant effect of stress compared to controls (*** p<0.001) and this was significantly reversed by administration of MYM-IV-46 at 10 mg/kg ($$<0.01). At 30 mg/kg MYM-IV-46 there was a trend level effect on alternation rate († p<0.1), there was no significant effect at 3 mg/kg (FIG. 19). These results suggest a potential indication of this compound to reverse cognitive deficits.

### Effects of Alpha PAMs in Other Cognitive Tests

Procedure for the Morris water maze assay: The potential of compounds to affect cognitive performance of rats may be assessed in 2 well-validated behavioral models. First, Morris water maze experiments are performed in a 2 m diameter circular pool filled to a height of 30 cm with water at 22±1° C. The escape platform (15 cm×10 cm) is submerged 2 cm below the water surface. All experimental details are as described in Savic et al. (Int. J. Neuropsychopharmacol. 2009, 12, 1179-1193). On each of the five consecutive days rats are given one swimming block, consisting of four trials. For each trial the rat is placed in the water at one of four pseudo-randomly determined starting positions. Once the rat has found and mounted the escape platform it is permitted to remain on the platform for 15 s. The rat is guided to the platform by the experimenter if it fails to locate it within 120 s. During the acquisition phase, treatments are applied once daily before the swimming block. On the sixth day, rats are given a treatment-free probe test (60 s) without the platform. The probe test is started from the novel, most distant location. Dependent variables chosen for tracking during the acquisition trials are: escape latency (s), total distance traveled (in), path efficiency (the ratio of the shortest possible path length to actual path length), % of distance swam in the peripheral annulus and mean speed (m/s). The selected parameters in the probe test are the distance swam in the target zone (s) and % of the distance swam in the peripheral annulus. 2 mg/kg diazepam is control drug. The doses of compounds are selected so that they elicit a mild, moderate and strong positive modulation of αC5GABAARs, respectively, in accordance with the analysis of thorough pharmacokinetic and electrophysiological data presented in Stamenic et al. (Eur. J. Pharmacol. 2016, 791, 433-443). The data from the acquisition days in the Morris water maze are averaged for each rat (total data/total number of trials per day) and analyzed using two-way ANOVA with repeated measures (factors: Treatment and Days) with Days as the repeated measure. In the case of significant interaction, separate one-way ANOVAs are conducted to assess the influence of treatment within individual levels of factor Days. The data from the probe test are assessed using one-way ANOVA.

Procedure for the social novelty discrimination (SND) assay: The SND test compares the social investigation times of an adult rat with a familiar and a novel juvenile rat. Testing consists of two consecutive juvenile presentations periods to an adult subject: period 1 (P1) and period 2 (P2). At the beginning of P1, one juvenile is placed into the adult home cage and the time spent by the adult investigating the juvenile (anogenital sniffing, licking, close pursuing and pawing) is recorded manually for 5 min. During P2, the same juvenile and a second, novel juvenile are placed in the cage together with the adult, and the times spent by the adult investigating each juvenile are measured independently for 3 min. A different pair of juvenile rats is presented to each adult tested. Manual scoring is conducted in a blinded manner. SND was deliberately impaired in control rats by the parametric manipulation applied, as there is 30 min delay between P1 and P2, and their SND was expected to be low. The influence of compounds on the thus induced impairment in SND is examined. There are five groups of rats which receive one of the following treatments 20 min before P1: solvent, 1.5 mg/kg diazepam or 1, 2.5 and 10 mg/kg test compound. The amount of time investigating familiar (Tf) and novel (Tn) juvenile during P2 is manually scored, and discrimination indexes (Tn−Tf/Tn+Tf) are calculated. Total exploration time during P1 and P2 is also manually recorded.

### Forced Swim Test

### Procedure for the Forced Swim Test Assay (FST)

Animals: C57/Bl6 male mice at 8 weeks-old are group housed (4-5 mice/cage) Linder normal housing conditions (12 h light ON cycle starting at 7 am/water and food ad libitum) for one week. During this week, animals are handled in order to habituate them to the experimenter and to reduce their anxiety-like response during behavioral testing.

Drug preparation and administration: All drugs are diluted in a vehicle solution containing 85% H₂O, 14% propylene glycol (Sigma Aldrich) and 1% Tween 80 (Sigma Aldrich). Working solutions are prepared at a concentration of 20 mg/mL and administered i.p. adjusted to the body weight of each animal. Doses used are either 1, 5 or 10 mg/kg. For all FST experiments, animals are injected i.p. with vehicle or test compound.

Protocol: Mice were tested in the forced swim test (FST) (measure of despair-like behavior used for the assessment of antidepressant efficacy). Animals are sub-chronically injected three times (24, 20 and 1 hour before testing) as per standard methods in the field for testing potential antidepressant compounds.

One hour after the last injection, mice are placed in an inescapable transparent tank filled with water (25 cm, 25-26&C), where they are unable to touch the bottom and unable to jump out of the tank. Animal are recorded for a period of 6 minutes and a manual count of the immobile time in the tank is measured for the 2-6 minutes period by an experimenter blinded to the mouse treatment history. Immobility is defined as the minimum amount of movement to stay afloat. Compounds that reduced immobility in the FST are considered to have potential antidepressant actions.

### Elevated Plus Maze (EPM)

The EPM is commonly used in preclinical rodent models to assess anxiety-like behaviors. Briefly, mice are placed in a cross-shaped maze raised at 55 cm from the floor, with 2 open arms facing each other, and 2 closed arms facing each other. During testing, mice are individually placed in the center of the maze facing an open arm and allowed to explore for 10 minutes. Throughout testing, the exploration of each mouse is recorded using a digital camera mounted on the ceiling. After 10 minutes of exploration, the camera is stopped and the mouse is removed from the maze and returned to its home cage. The videos are analyzed using Ethovision XT14 software. Specific parameters considered are: % time spent in the open arms, calculated using ([(total time spent in open arms)/(total time spent in open arms+total time spent in closed arms)]*100) and the number of open arm entries.

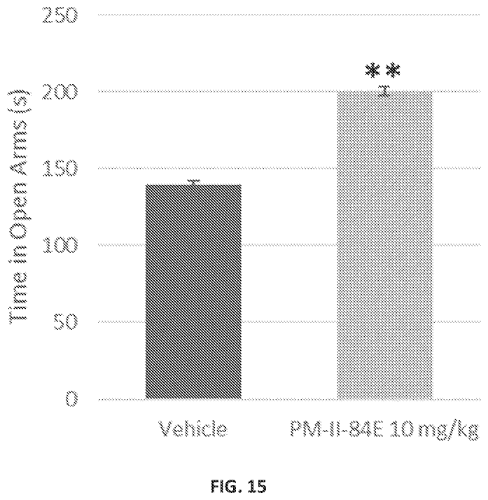

Results in the EPM with PM-II-84E. Mice treated with PM-II-84E (10 mg/kg, i.p., 30 min before testing) and vehicle controls were tested in the elevated plus maze for anxiety like behaviors. t-tests found a significant effect of PM-II-84E on percent of entries to the open arm (* p<0.05) and percent of time spent in the open arm (** p<0.01) and a trend level effect on percent of distance travelled in the open arm († p<0.10) (n=6 per group), suggesting anxiolytic properties (FIG. 15).

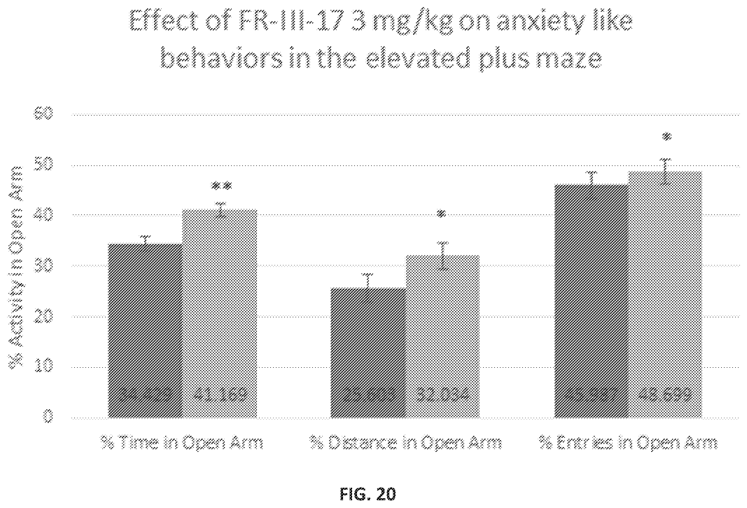

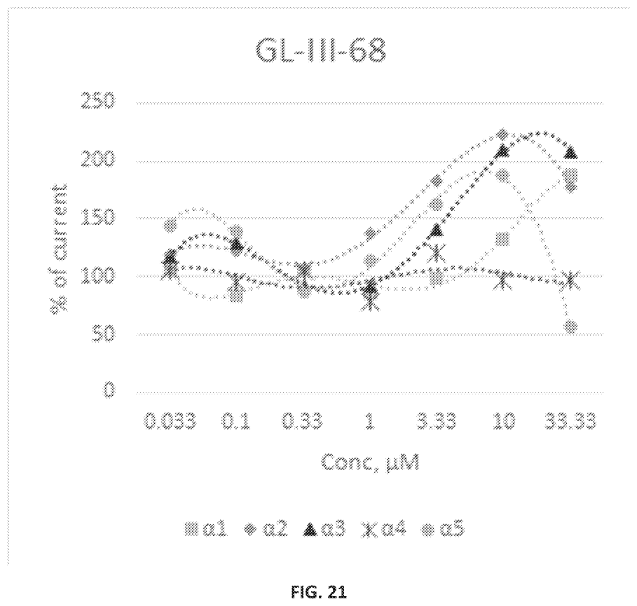

Results in the EPM with FR-III-17. Mice treated with FR-III-17 (3 mg/kg, i.p., 30 min before testing) and vehicle controls were tested in the elevated plus maze for anxiety like behaviors. t-tests found FR-III-17 significantly increase % time, % distance traveled and % entries in the open arms (* p<0.05, ** P<0.01) (n=6 per group), suggesting anxiolytic properties (FIG. 20).

### Locomotor Activity

Home cage locomotor activity changes for test compounds may be quantified to assess the animal's movement, in dim light condition and to detect potential sedative effects of the different compounds. Mice are placed in a clean cage, similar to their home-cage (28.2×17.1 cm), without bedding and lid to allow video recording from the top. Tacking is performed and distance travelled (30 min session) is analyzed using ANY-Maze™ tracking software (version 499z) to assess the locomotor activity in a home-cage environment. Animals receive a single dose of compound or vehicle solution 1 hour before testing.

Drug preparation and administration: All drugs are diluted in a vehicle solution containing 85% H2O, 14% propylene glycol (Sigma Aldrich) and 1% Tween 80 (Sigma Aldrich). Working solutions are prepared at a concentration of 20 mg/mL and administered i.p. adjusted to the body weight of each animal. Doses used are either 1, 5 or 10 mg/kg. For all locomotor activity experiments, animals are acutely injected i.p. with vehicle or test compound, 1 hour before the beginning of the test.

### Motor Coordination

Rotarod test is performed on mouse rotarod (Ugo Basile, Comerio, Italy) in order to observe the capacity of the animal to maintain itself on the rod revolving 15 rpm. The day before testing, mice are trained in three sessions in a row with each session lasting 180 s and at least 30 minute pauses between. On the next day, selection is made and mice fulfilling the criteria of maintaining themselves for 180 s on the rod without falling off are included in the test which started two hours later. Male C57BL/6 mice are tested 20 minutes, 1 and 3 hours after peroral application of the solvent (SOL) or compound. Latency to fall from the rotarod is recorded manually by experimenter. Dosage forms of the compounds are prepared by diluting/suspending them in the SOL (85% distilled water, 14% propylene glycol and 1% Tween 80) with the aid of sonication. An appropriate volume of the treatments are applied by intragastric probe. Graphic interpretation of the experiment is performed in Sigma plot 12 (Systat, USA).

### Electrophysiology

Procedure A: Transfection of Mammalian Cells and Electrophysiological Recordings. Full-length cDNAs for GABA_{A} receptor subtypes (generously provided by Dr. Robert Macdonald, Vanderbilt University and Dr. David Weiss, University of Texas Health Science Center, San Antonio, TX) in mammalian expression vectors were transfected into the human embryonic kidney cell line HEK-293T (GenHunter, Nashville, TN) (Chestnut et al, 1996, J. Immunol. Methods 193, 17-27). All subtypes were rat clones except for α2, which was a human clone. Cells were maintained in Dulbecco's modified Eagle's medium (DMEM) plus 10% fetal bovine serum, 100 IU/mL penicillin, and 100 μg/mL streptomycin.

HEK-293T cells were transiently transfected using calcium phosphate precipitation. Plasmids encoding GABA_{A} receptor subtype cDNAs were added to the cells in 1:1:1 ratios (α:β:γ) of 2 μg each.31 For identification of positively transfected cells, 1 μg of the plasmid pHook-1 (Invitrogen Life Technologies, Grand Island NY) containing cDNA encoding the surface antibody sFv was also transfected into the cells.11 Following a 4-6 h incubation at 3% CO2, the cells were treated with a 15% glycerol solution in BBS buffer (50 mM BES(N,N-bis[2-hydroxyethyl]-2-aminoethanesulfonic acid), 280 mM NaCl, 1.5 mM Na2HPO4) for 30 s. The selection procedure for pHook expression was performed 18-52 h later. The cells were passaged and mixed for 30-60 min with 3-5 μL of magnetic beads coated with antigen for the pHook antibody (approximately 6×105 beads).11 Bead-coated cells were isolated using a magnetic stand. The selected cells were resuspended into supplemented DMEM, plated onto glass coverslips treated with poly L-lysine and collagen, and used for recordings the next day.

Cells are patch-clamped at −50 mV in the whole-cell recording configuration. The bath solution consists of (in mM): 142 NaCl, 8.1 KCl, 6 MgCl2, 1 CaCl2, and 10 HEPES (4-(2-hydroxyethyl)-1-piperazineethanesulfonic acid) with pH=7.4 and osmolarity adjusted to 295-305 mOsm. Recording electrodes are filled with a solution of (in mM) 153 KCl, 1 MgCl2, 5 K-EGTA (ethylene glycol-bis(β-aminoethyl ether N,N,N′N′-tetraacetate), and 10 HEPES with pH=7.4 and osmolarity adjusted to 295-305 mOsm. GABA is diluted into the bath solution from freshly made or frozen stocks in water. Compounds are dissolved in DMSO and diluted into bath solution with the highest DMSO level applied to cells of 0.01%. Patch pipettes are pulled from borosilicate glass (World Precision Instruments, Sarasota, FL) on a two-stage puller (Narishige, Japan) to a resistance of 5-10 Mo. Solutions containing GABA or GABA+ compounds are applied to cells for 5 s using a 3-barreled solution delivery device controlled by a computer-driven stepper motor (SF-77B, Harvard Apparatus, Holliston, MA, open tip exchange time of <50 ms). There is a continuous flow of external solution through the chamber. Currents are recorded with an Axon 200B (Foster City, CA) patch clamp amplifier.

Whole-cell currents are analyzed using the programs Clampfit (pClamp9 suite, Axon Instruments, Foster City, CA) and Prism (Graphpad, San Diego, CA). Concentration-response data is fit with a four-parameter logistic equation (current=[minimum current+(maximum current−minimum current)]/1+(10(log EC50−log[modulator])n) where n represents the Hill number. All fits are made to normalized data with current expressed as a percentage of the response to GABA alone for each cell.

Procedure B: Full-length cDNAs for GABA_{A} receptor subtypes are transfected into the human embryonic kidney cell line HEK-293T. All subtypes are rat clones except for α2, which is a human clone. Cells are patch-clamped at −50 mV in the whole-cell recording configuration. GABA is diluted into the bath and compounds are dissolved in DMSO and diluted into bath solution. Solutions containing GABA or GABA+ compounds are applied to cells for 5 s. Currents are recorded with an Axon 200B patch clamp amplifier. Data is normalized data current expressed as a percentage of the response to GABA alone for each cell.

Electrophysiological Characterization of GL-III-68 by Procedure B: Human mammalian cells are transfected with human α1β2γ3, α2β2γ3, α3β2γ3, α4β2γ3, or α5β2γ3 GABA_{A} receptors. Cells are incubated with GL-III-68 at a range of concentrations from 0.03 μM to 100 μM. Currents are induced by stimulation with 5 μM GABA and recorded using the IonWorks Barracuda® test platform. Current is normalized to the current induced by 5 μM of GABA in the absence of any allosteric modulator. PAM effect of at different subtypes of GABA^{A} receptor. % of current was calculated relatively to current produced by 5 μM GABA alone (set to 100%) (FIG. 21). EC₅₀ results are shown below.

EC₅₀ of GL-III-68 at α1, α2, α3, α4 or α5 in Mammalian Cells Transfected with α1, α2, α3, α4 α5, or Receptor

| α1β3γ2 | | α2β3γ2 | | α3β3γ2 | | α4β3γ2 | | α5β3γ2 | |
|---|---|---|---|---|---|---|---|---|---|
| E_{MAX}, % | EC₅₀, μM | E_{MAX}, % | EC₅₀, μM | E_{MAX}, % | EC₅₀, μM | E_{MAX}, % | EC₅₀, μM | E_{MAX}, % | EC₅₀, μM |
| 188 | 10.67 | 224 | 1.91 | 209 | 3.52 | — | — | 188 | <0.03 |

What is claimed is:

1. A compound selected from the group consisting of: 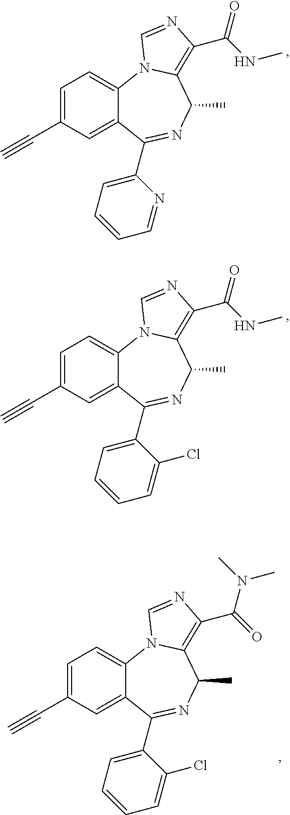 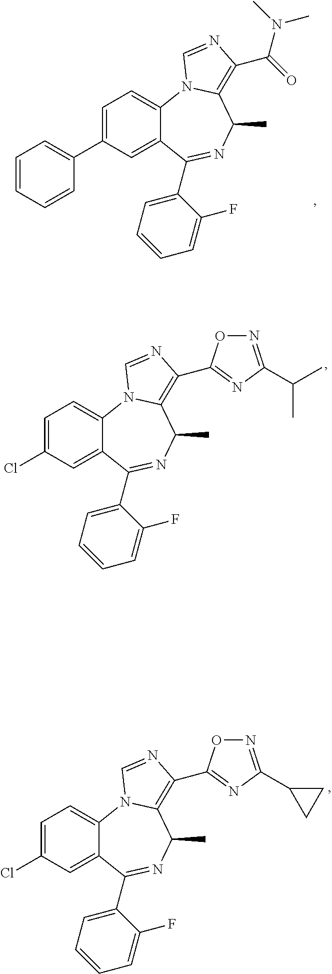 [SMILES: C[C@H]1N=C(c2ccccc2F)c2cc(-c3ccccc3)ccc2-n2cnc(C(=O)N(C)C)c21 ; CC(C)c1noc(-c2ncn3c2[C@@H](C)N=C(c2ccccc2F)c2cc(Cl)ccc2-3)n1 ; C[C@H]1N=C(c2ccccc2F)c2cc(Cl)ccc2-n2cnc(-c3nc(C4CC4)no3)c21] 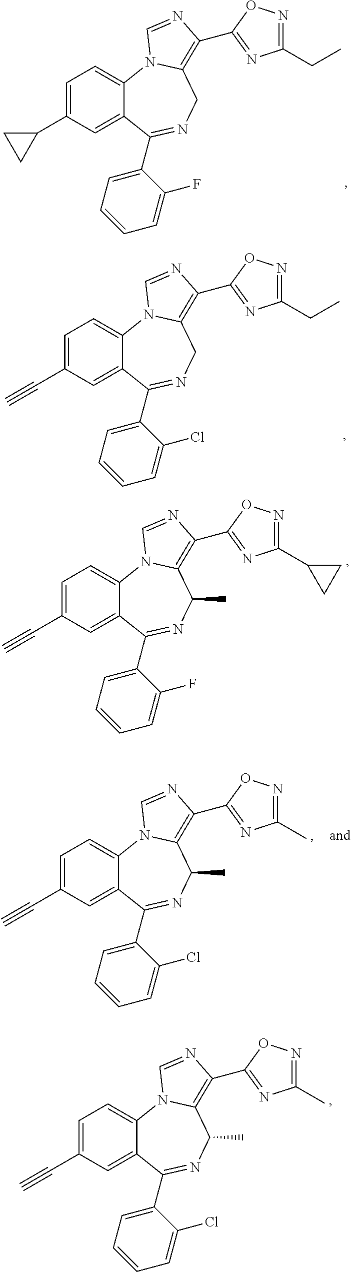
or a pharmaceutically acceptable salt thereof.

2. The compound of claim 1, or a pharmaceutically acceptable salt thereof, wherein the compound is

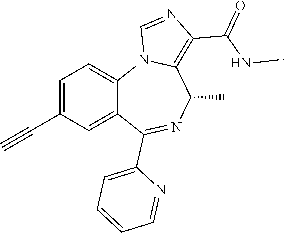 [SMILES: C#Cc1ccc2c(c1)C(c1ccccn1)=N[C@@H](C)c1c(C(=O)NC)ncn1-2]

3. The compound of claim 1, or a pharmaceutically acceptable salt thereof, wherein the compound is

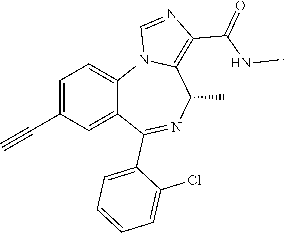 [SMILES: C#Cc1ccc2c(c1)C(c1ccccc1Cl)=N[C@@H](C)c1c(C(=O)NC)ncn1-2]

4. The compound of claim 1, or a pharmaceutically acceptable salt thereof, wherein the compound is

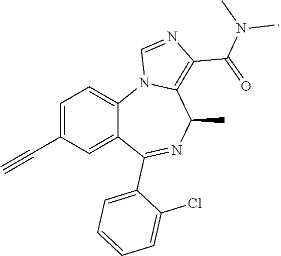 [SMILES: C#Cc1ccc2c(c1)C(c1ccccc1Cl)=N[C@H](C)c1c(C(=O)N(C)C)ncn1-2]

5. The compound of claim 1, or a pharmaceutically acceptable salt thereof, wherein the compound is

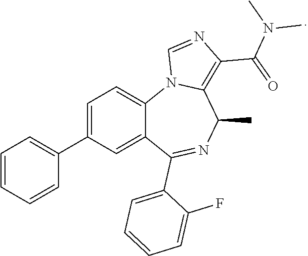 [SMILES: C[C@H]1N=C(c2ccccc2F)c2cc(-c3ccccc3)ccc2-n2cnc(C(=O)N(C)C)c21]

6. The compound of claim 1, or a pharmaceutically acceptable salt thereof, wherein the compound is

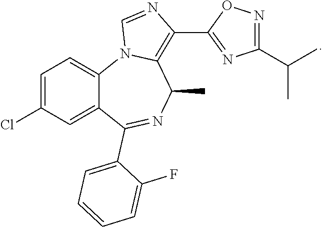 [SMILES: CC(C)c1noc(-c2ncn3c2[C@@H](C)N=C(c2ccccc2F)c2cc(Cl)ccc2-3)n1]

7. The compound of claim 1, or a pharmaceutically acceptable salt thereof, wherein the compound is

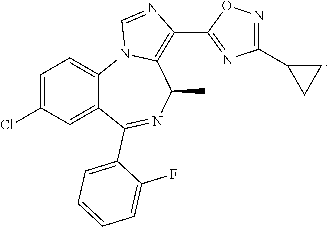 [SMILES: C[C@H]1N=C(c2ccccc2F)c2cc(Cl)ccc2-n2cnc(-c3nc(C4CC4)no3)c21]

8. The compound of claim 1, or a pharmaceutically acceptable salt thereof, wherein the compound is

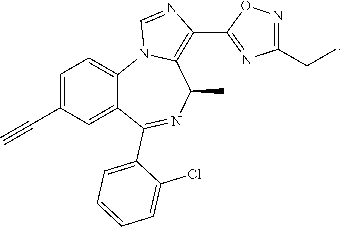 [SMILES: C#Cc1ccc2c(c1)C(c1ccccc1Cl)=N[C@H](C)c1c(-c3nc(CC)no3)ncn1-2]

9. The compound of claim 1, or a pharmaceutically acceptable salt thereof, wherein the compound is

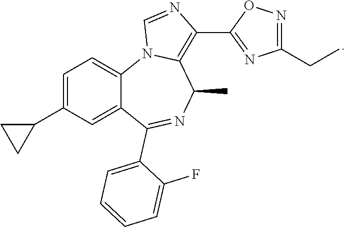 [SMILES: CCc1noc(-c2ncn3c2[C@@H](C)N=C(c2ccccc2F)c2cc(C4CC4)ccc2-3)n1]

10. The compound of claim 1, or a pharmaceutically acceptable salt thereof, wherein the compound is

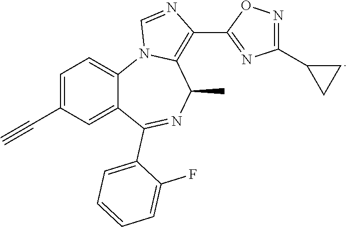 [SMILES: C#Cc1ccc2c(c1)C(c1ccccc1F)=N[C@H](C)c1c(-c3nc(C4CC4)no3)ncn1-2]

11. The compound of claim 1, or a pharmaceutically acceptable salt thereof, wherein the compound is

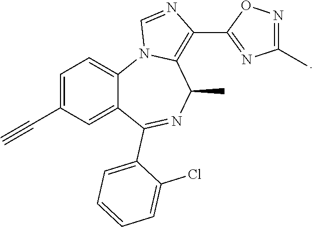 [SMILES: C#Cc1ccc2c(c1)C(c1ccccc1Cl)=N[C@H](C)c1c(-c3nc(C)no3)ncn1-2]

12. The compound of claim 1, or a pharmaceutically acceptable salt thereof, wherein the compound is

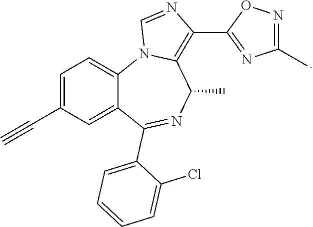 [SMILES: C#Cc1ccc2c(c1)C(c1ccccc1Cl)=N[C@@H](C)c1c(-c3nc(C)no3)ncn1-2]

13. The compound of any of claims 1-8 or 10-12, or a pharmaceutically acceptable salt thereof, wherein the compound is substantially enantiomerically pure.

14. A pharmaceutical composition comprising the compound of any of claims 1-13, or a pharmaceutically acceptable salt thereof, and a pharmaceutically acceptable carrier.

15. A method of enhancing cognition comprising administering to a subject in need thereof a therapeutically effective amount of the compound of any of claims 1-13, or the pharmaceutical composition of claim 14, or a compound selected from the group consisting of: 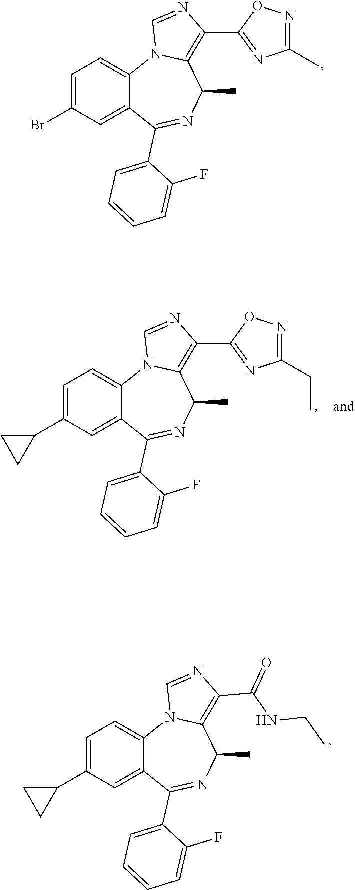 [SMILES: Cc1noc(-c2ncn3c2[C@@H](C)N=C(c2ccccc2F)c2cc(Br)ccc2-3)n1 ; CCc1noc(-c2ncn3c2[C@@H](C)N=C(c2ccccc2F)c2cc(C4CC4)ccc2-3)n1 ; CCNC(=O)c1ncn2c1[C@@H](C)N=C(c1ccccc1F)c1cc(C3CC3)ccc1-2]
or a pharmaceutically acceptable salt or composition thereof.
